(19) United States
(12) Patent Application Publication
(10) Pub. No.: US 2024/0277281 A1
(43) Pub. Date: Aug. 22, 2024

(54) USER ASSISTANCE SYSTEM, WEARABLE TERMINAL, AND USER ASSISTANCE METHOD

(71) Applicant: MAXELL, LTD., Kyoto (JP)

(72) Inventors: Mayumi NAKADE, Kyoto (JP); Yasunobu HASHIMOTO, Kyoto (JP); Hitoshi AKIYAMA, Kyoto (JP)

(21) Appl. No.: 18/570,581

(22) PCT Filed: Jun. 15, 2021
(86) PCT No.: PCT/JP2021/022692

§ 371 Date: Dec. 14, 2023

(51) Int. Cl. A61B 5/369; A61B 5/00; G08B 21/04; G16H 40/60

(52) U.S. Cl. CPC A61B 5/369; A61B 5/4809; G08B 21/0453; G16H 40/60

(57) ABSTRACT

A user assistance system comprises an electroencephalogram measurement device configured to output electroencephalogram data on a user, an action measurement device configured to output action data on the user, an electroencephalogram determination device configured to determine whether an abnormality is found in the electroencephalogram data based on the electroencephalogram data and the action data, and a controller configured to carry out predetermined processing in response to a result of determination indicating that the abnormality has been found.

### CROSS-REFERENCE TO RELATED APPLICATIONS

This application is the U.S. National Phase under 35 U.S.C. § 371 of International Application No. PCT/JP2021/022692, filed on Jun. 15, 2021, the entire contents of which are hereby incorporated by reference.

### TECHNICAL FIELD

The present invention relates to a user assistance system. In particular, the present invention relates to a system for assisting a user who is determined to have an abnormality in his or her electroencephalogram.

### BACKGROUND ART

The prevalence of dementia is over 15% in the elderly people aged 65 years and over, and in this aging society, dementia is not rare disease. In recent years, treatment and medications have been developed, and it has been proven that early detection and treatment can suppress the progression of dementia.

It is known that diagnosis of dementia can be made by electroencephalogram measurement. There has been known a system using this to determine the potential risk of dementia. For example, Patent Literature 1 discloses a system including: “a physiological data detection sensor for physiological data of a subject during sleeping; a sleep data generation device for generating sleep data including the depth of sleep and the change in body motion of the subject with the lapse of time based on the physiological data of the subject acquired by the physiological data detection sensor; and a dementia risk determination device equipped with a storage for storing sleep data specific to each symptom of dementia obtained from real patients, the dementia risk determination device being configured to compare the sleep data of the subject generated by the sleep data generation device with the sleep data of each symptom stored in the storage to determine three dementia risks based on the sleep data of the subject.

Furthermore, Patent Literature 2 discloses a head-mounted electroencephalogram measurement device.

### CITATION LIST

### Patent Literature

Patent Literature 1: JP-A-2016-22310
Patent Literature 2: WO2020/150193

### SUMMARY OF INVENTION

### Technical Problem

As described above, progression of dementia can be controlled by early detection. However, in many cases, a patient takes electroencephalogram measurement and dementia examination in a hospital or the like after an abnormality in his or her daily behavior is found. Furthermore, a person who lives alone and has less contact with other people does not take an examination in a hospital unless he or she doubts the possibility of dementia by himself or herself, and this may lead to progression of dementia.

According to the technique disclosed in Patent Literature 1, determination is made based on physiological data during sleeping. However, symptoms of dementia are not always be found during sleeping, and thus early detection of dementia cannot be always possible according to the disclosed technique. Furthermore, in the technique disclosed in Patent Literature 1, no countermeasure is proposed even if a user is determined to have dementia. Note that symptoms in which whether an abnormality is found can be detected based on an electroencephalogram are not limited to the symptoms of dementia.

The present invention has been made in view of the circumstances described above, and an object of the present invention is to provide a technique capable of responding to an abnormality such as dementia with a simple configuration without imposing a burden on a user.

### Solution to Problem

In order to achieve the object, provided is a user assistance system comprising: an electroencephalogram measurement device configured to output electroencephalogram data on a user; an action measurement device configured to output action data on the user; an electroencephalogram determination device configured to determine whether an abnormality is found in the electroencephalogram data based on the electroencephalogram data and the action data; and a controller configured to carry out predetermined processing in response to a result of determination indicating that the abnormality has been found.

### Advantageous Effects of Invention

According to the present invention, it is possible to respond to an abnormality such as dementia with a simple configuration without imposing a burden on a user. The problems, configurations, and advantageous effects other than those described above will be clarified by explanation of the embodiments below.

### BRIEF DESCRIPTION OF DRAWINGS

FIG. 1A is a diagram for explaining an outline of processing according to the first embodiment.

FIG. 1B is a diagram for explaining an outline of processing according to the first embodiment.

FIG. 2A illustrates the appearance of a head mounted display (HMD) according to the first embodiment.

FIG. 2B is a diagram for explaining a state in which the HMD is being worn.

FIG. 3 is a hardware configuration diagram of the HMD according to the first embodiment.

FIG. 4 is a functional block diagram of the HMD according to the first embodiment.

FIG. 5A is a diagram for explaining examples of action state data, electroencephalograms for comparison, and abnormality information according to the first embodiment.

FIG. 5B is a diagram for explaining examples of action state data, electroencephalograms for comparison, and abnormality information according to the first embodiment.

FIG. 5C is a diagram for explaining examples of action state data, electroencephalograms for comparison, and abnormality information according to the first embodiment.

FIG. 6A is a diagram for explaining a display example according to the first embodiment and a display example according to a modification.

FIG. 6B is a diagram for explaining a display example according to the first embodiment and a display example according to a modification.

FIG. 6C is a diagram for explaining a display example according to a modification of the third embodiment.

FIG. 7 illustrates a flowchart of analysis and evaluation processing according to the first embodiment.

FIG. 8 illustrates a flowchart of analysis and evaluation processing according to a modification of the first embodiment.

FIG. 9A is a diagram for explaining usage modes according to different modifications of the first embodiment.

FIG. 9B is a diagram for explaining usage modes according to different modifications of the first embodiment.

FIG. 10 is a functional block diagram of an HMD according to the second embodiment.

FIG. 11A is a diagram for explaining an example of an instruction setting according to the second embodiment. FIG. 11B is a diagram for explaining display examples according to the second embodiment.

FIG. 11C is a diagram for explaining display examples according to the second embodiment.

FIG. 12 illustrates a flowchart of analysis and evaluation processing according to the second embodiment.

FIG. 13 is a diagram for explaining a modification according to the second embodiment.

FIG. 14A is a diagram for explaining an instruction setting and usage modes according to a modification of the second embodiment.

FIG. 14B is a diagram for explaining an instruction setting and usage modes according to a modification of the second embodiment.

FIG. 15 is a diagram for explaining transmission and reception of signals between an HMD and a server according to a modification of the second embodiment.

FIG. 16 is a functional block diagram of an HMD according to the third embodiment.

FIG. 17A is a diagram for explaining an example of an assistance application database and an example of output according to the third embodiment.

FIG. 17B is a diagram for explaining an example of an assistance application database and an example of output according to the third embodiment.

FIG. 18 illustrates a flowchart of analysis and evaluation processing according to the third embodiment.

FIG. 19 illustrates a flowchart of analysis and evaluation processing according to a modification of the third embodiment.

FIG. 20 illustrates a flowchart of analysis and evaluation processing according to the fourth embodiment.

FIG. 21A is a diagram for explaining the appearance of a smartphone which is a modification according to embodiments of the present invention.

FIG. 21B is a diagram for explaining the appearance of a smartphone which is a modification according to embodiments of the present invention.

FIG. 21C is a diagram for explaining the appearance of a smartphone which is a modification according to embodiments of the present invention.

### DESCRIPTION OF EMBODIMENTS

Hereinafter, embodiments of the present invention will be described with reference to the drawings. Each embodiment of the present invention, which realizes responding to diseases such as dementia, contributes to “Goal 3: Ensure healthy lives and promote well-being for all at all ages” of the Sustainable Development Goals (SDGs) advocated by the United Nations.

### First Embodiment

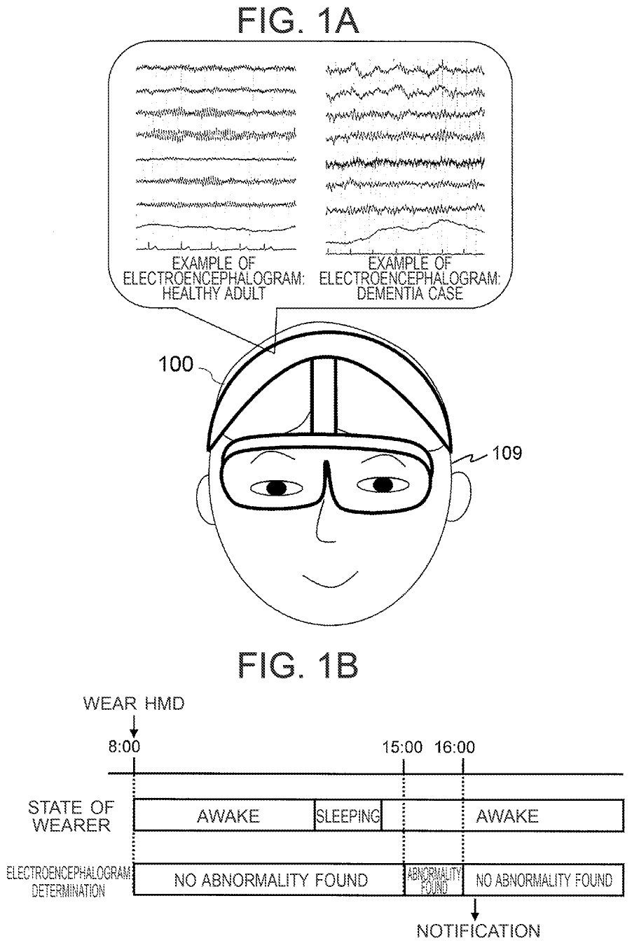

The first embodiment of the present invention will be described. Firstly, an outline of the present embodiment will be described with reference to FIG. 1A and FIG. 1B.

In the present embodiment, a function of measuring and analyzing an electroencephalogram is provided in an HMD (Head Mounted Display) which is routinely used by a user. When it is found that the user is suspected to have dementia, the user is notified of the possibility of dementia or the like while the user's consciousness is clear so that an early examination and treatment can be encouraged to the user. This contributes to the early detection of dementia or the like, and as a result, it is possible to delay the progression of the disease.

Specifically, as illustrated in FIG. 1A, an HMD 100 equipped with a function of measuring an electroencephalogram is used. The HMD 100 detects an action of a wearer (user) 109, determines whether the wearer is awake (during awake) or asleep (during sleep), and detects the electroencephalogram of the wearer 109 to determine whether the possibility of an abnormality is found.

For example, for determining whether the possibility of dementia as an abnormality is found, as illustrated in FIG. 1A, for example, the HMD 100 stores an electroencephalogram of a healthy adult and an electroencephalogram of an adult with dementia in advance. The HMD 100 compares the electroencephalograms with the electroencephalogram as detected to determine whether an abnormality (suspicion of dementia) is found.

Hereinafter, in the present specification, the case that an abnormality may be suspected is simply referred to as “an abnormality is found (abnormal)”. On the other hand, the case that an abnormality is not suspected is referred to as “no abnormality is found (not abnormal)”. In the present embodiment, dementia will be exemplified as an abnormality to be detected. Note that the disease (symptom) to which the present embodiment is applicable is not limited to dementia as long as a state of “an abnormality is found” and a state of “no abnormality is found” appear in the electroencephalogram.

As illustrated in FIG. 1B, upon determining “an abnormality is found”, the HMD 100 temporarily records the determination. Then, at the timing when determining that the wearer 109 is awake and also determining “no abnormality is found”, the HMD 100 notifies the wearer 109 of the abnormality being found. At this time, the HMD 100 may further provide a stimulus to the wearer 109 (moving a light, making questions) to make the determination more accurate.

As described above, in the present embodiment, the HMD 100 is configured to determine whether an abnormality is found in the wearer 109 using his or her electroencephalogram, and notifies the wearer 109 of the determination at the timing when determining “no abnormality is found”. That is, the HMD 100 according to the present embodiment notifies the wearer 109 of suspicion of dementia and encourages him or her to go to the hospital, for example, while the degree of dementia of the wearer 109 is low. This enables, for example, early detection of an abnormality in the brain such as dementia, and as a result, the wearer 109 can be made aware of the abnormality.

Hereinafter, the HMD 100 according to the present embodiment for realizing the functions described above will be described.

### [Appearance]

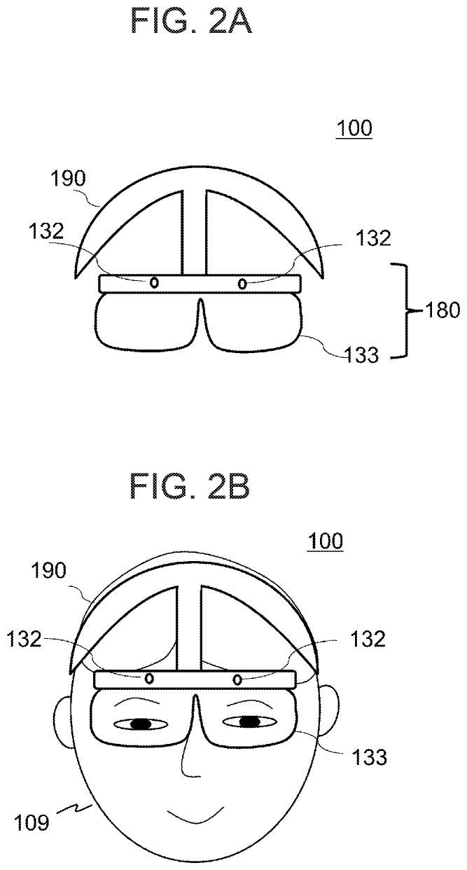

The appearance of the HMD 100 according to the present embodiment will be described with reference to FIG. 2A and FIG. 2B. FIG. 2A illustrates the appearance of the HMD 100, and FIG. 2B illustrates the state in which the wearer 109 is wearing the HMD 100.

As illustrated in FIG. 2A, the HMD 100 includes a main body portion 180 and a support portion 190 that supports the main body portion 180.

The main body 180 is provided with, for example, a display 133, an in-camera 131, an out-camera 132, a configuration (information processing unit) for realizing an information processing function, and the like. The hardware configuration of the main body 180 will be described later.

The support portion 190 is coupled to the main body portion 180, and engages the head of the wearer 109 to support the main body portion 180. As illustrated in FIG. 2B, the main body portion 180 is supported by the support portion 190 on the head of the wearer 109. The support portion 190 is, for example, a hair band type as illustrated in FIG. 2B. The support portion 190 may have, for example, the shape of a band extending to surround the side surfaces and the rear surface of the head of the wearer 109.

Furthermore, in the present embodiment, a plurality of electrodes placed on the head of the wearer 109 is used to acquire an electroencephalogram based on the potential difference. The plurality of electrodes is placed on the main body portion 180 and/or the support portion 190. The placement positions are determined depending on the methods of acquiring an electroencephalogram. Details will be described later.

As the electrodes, for example, commonly used disk electrodes are used. The electrodes are made of silver, silver chloride, or the like. Note that a silicone-based conductive rubber may be used.

### [Hardware Configuration]

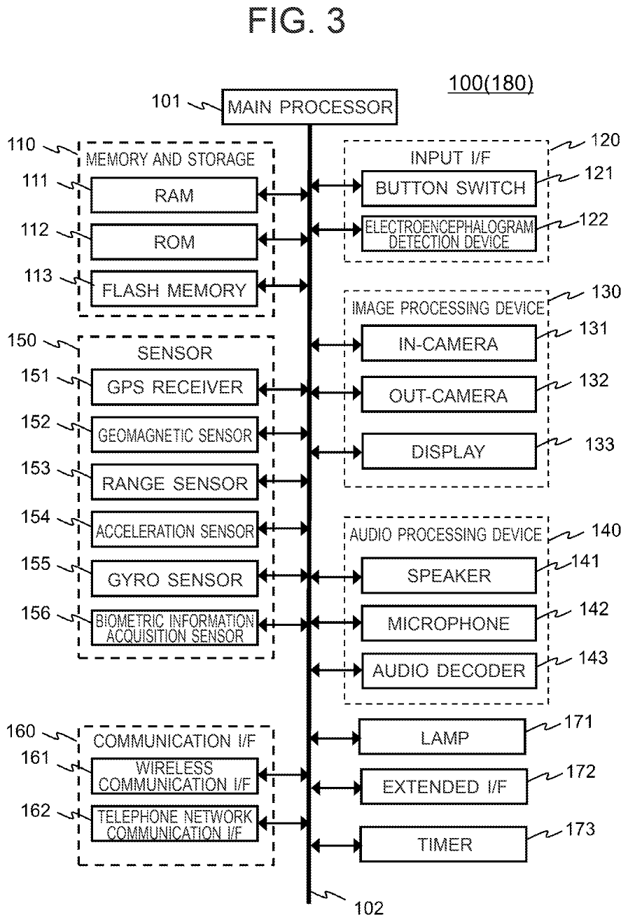

Next, the hardware configuration of the main body portion 180 of the HMD 100 will be described. FIG. 3 is a hardware configuration diagram of the main body portion 180 according to the present embodiment. As illustrated in FIG. 3, the main body portion 180 of the HMD 100 includes a main processor 101, a system bus 102, a memory and storage 110, an input interface (I/F) 120, an image processing device 130, an audio processing device 140, a sensor 150, and a communication interface (I/F) 160.

The main processor 101 is a main controller for controlling the entire of the HMD 100 in accordance with a predetermined program. The main processor 101 is implemented by a CPU (Central Processor Unit) or a microprocessor unit (MPU). The main processor 101 executes the processing in accordance with a clock signal which is measured and output by a timer equipped in the HMD 100.

The main processor 101 reads out and executes, in a working area of a RAM 111, for example, an operating system (OS) stored in the memory and storage 110 and various application programs for operation control, thereby controlling each part of the HMD 100 and realizing the OS and each function.

The system bus 102 is a data communication channel for transmitting and receiving data between the main processor 101 and each unit of the main body portion 180 of the HMD 100.

The memory and storage 110 stores data necessary for the processing performed by the main processor 101, data generated by the processing, and the like. The memory and storage 110 includes the RAM (Random Access Memory) 111, a ROM (Read Only Memory) 112, and a flash memory 113.

The RAM 111 is a program area used for execution of a basic operation program and other application programs. The RAM 111 is a temporary storage area for temporarily storing data as needed during execution of various application programs. The RAM 111 may be integrated with the main processor 101.

The ROM 112 and the flash memory 113 store operation setting values of the HMD 100, information about the user (wearer 109) of the HMD 100, and the like. They may store data such as still image data and moving image data which are captured by the HMD 100. The functions of the HMD 100 can be expanded by downloading a new application program from an application server through the Internet. At this time, the downloaded new application program is stored in the ROM 112 and the flash memory 113. The main processor 101 loads and executes, in the RAM 111, the new application program stored therein, thereby realizing various functions. Instead of the ROM 112 and the flash memory 113, devices such as an SSD (Solid State Drive) and an HDD (Hard Disc Drive) may be used.

The main processor 101 expands and executes programs stored in the ROM 112 and the flash memory 113, in the RAM 111, whereby each function of the information processing unit (computer), which will be described later, is also implemented.

The input I/F 120 receives an input to the HMD 100. The input I/F 120 includes, for example, a button switch 121 and an electroencephalogram detection device 122. In addition, the input I/F 120 may include operation keys such as a power key, a volume key, and a home key. The input I/F 120 may include a touch sensor for receiving an operation instruction via the touch pad. The touch sensor is placed as a touch panel so as to be superimposed on a display 133 which will be described later.

Furthermore, an operation to the HMD 100 may be accepted via a separate information processing terminal device connected to the HMD 100 by wired communication or wireless communication.

The electroencephalogram detection device 122 receives an electroencephalogram acquired by the electrodes.

The image processing device 130 includes an image (video) processor, and includes the in-camera 131, the out-camera 132, and the display 133.

Each of the in-camera 131 and the out-camera 132 captures a field of view of the surroundings, and converts a light incident from the lens into an electrical signal by an image sensor to obtain an image. The number of the in-camera 131 and out-camera 132 to be provided is not limited.

The out-camera 132 acquires an image around the HMD 100. The out-camera 132 is provided on a surface (front surface) of the main body portion 180 opposite to the screen of the display 133. As illustrated in FIG. 2A, the out-camera 132 is placed to face outward, for example, on the upper portion of the display 133 of the main body portion 180.

The in-camera 131 captures an image of the face or eyes of the wearer 109 seeing the display and takes the image into the HMD 100. The in-camera 131 functions as a line-of-sight detection sensor as well. The in-camera 131 is placed to face inward (toward the wearer 109), for example, on the upper portion of the display 133 of the main body portion 180. In other words, the in-camera 131 is provided on the surface of the main body portion 180, which is common screen to the display 133. The in-camera 131 may be arranged at any position as long as it allows the eyes to be imaged in order to detect the line-of-sight direction of the wearer 109 as described above. Furthermore, the in-camera 131 detects the opening and closing of the eyes of the wearer 109 as well.

The images acquired by the in-camera 131 and the out-camera 132 are processed by an image (video) signal processor or the main processor 101, and output to the display 133 with objects generated by the main processor 101 or the like are superimposed thereon.

The display 133 is a display device using, for example, a laser, a liquid crystal panel, an organic EL (EL: Emitting Diode), or the like. Causing the wearer 109 to directly see the display device through a lens or the like, see the image data projected onto the glasses, or see the image data projected onto the retina of the wearer 109 enables the image data processed by the image processor to be presented to the wearer 109 of the HMD 100. The display 133 shows notification information such as warning information and instruction information to the wearer 109, icons of applications, various state display images, and the like on the screen.

As illustrated in FIG. 2B, the display 133 is placed in front of both eyes of the wearer 109.

Note that the display 133 may be an optically transmissive (Optical See-Through) type display or a video transmissive (Video See-Through) type display. The optical transmissive display includes a half mirror and allows the wearer to see the surrounding states (real view) through the display screen. In the case of a video transmissive display, the wearer cannot see the real scene, but the surrounding states captured by the out-camera 132 is displayed on the display screen. The wearer 109 can recognize the external states by viewing the images thereof.

Furthermore, a touch panel may be laminated on the screen of the display 133. The touch panel is made of a touch panel member such as a capacitive touch panel, and allows the wearer 109 to input information desired to be input by operations such as approaching a finger, a touch pen, or the like or making them touch (hereinafter, referred to as touching). The touch panel is an example of an operation input member. The operation input member may be, for example, a keyboard or a key button which is connected via the communication I/F 160, the voice input device which collects, using the microphone 142, the voice of the wearer 109 indicating an input operation and converts it into operation information, or a gesture input device which captures an image of a gesture made by the wearer 109 and analyzes it.

The audio processing device 140 includes an audio signal processor that processes the audio data, and includes a speaker 141 serving as an audio output unit and a microphone 142 serving as an audio input unit. The speaker 141 may be, for example, two speakers and placed on the support portion 190 near the right ear and the left ear of the wearer 109, respectively. The microphone 142 is placed, for example, at a distal end of a microphone support portion extending from the support portion 190.

The speaker 141 issues various types of output information (including notification information and instruction information) in the HMD 100 to the outside by means of providing audio data. Furthermore, the microphone 142 collects the sounds from the outside or voices of the wearer 109, converts them into audio signals, and captures the audio signals into the HMD 100. The HMD 100 captures the operation information by means of sounds made by the wearer 109, thereby easily executing the operations corresponding to the operation information.

The sensor 150 is a group of sensors for detecting the state of the HMD 100. The sensor 150 includes, for example, a GPS (Global Positioning System) receiver 151, a geomagnetic sensor 152, a range sensor 153, an acceleration sensor 154, a gyro sensor 155, and a biometric information acquisition sensor 156. Note that the sensor 150 is not limited thereto, and does not necessarily include all of them. The HMD 100 detects the position, motion, tilting, direction, and the like of the HMD 100 using this group of sensors.

The sensors of the sensor 150 may be arranged at any position. However, the range sensor 153 is to be placed on the front surface of the HMD 100 to detect a distance from the HMD 100 to the object.

The communication I/F 160 is a communication processor for executing communication processing. The communication I/F 160 includes a wireless communication I/F 161 and a telephone network communication I/F 162. The wireless communication I/F 161 is an interface for wireless communication, and performs communication using a LAN (Local Area Network), near field wireless communication, and the like. In the case of LAN communication, the wireless communication I/F 161 is connected with an access point for wireless communication on the Internet by wireless communication to transmit and receive data. The telephone network communication I/F 162 performs telephone communication (telephone call) and transmission and reception of data by wireless communication with a base station of a mobile phone communication network. Each of the wireless communication I/F 161 and the telephone network communication I/F 162 includes an encoding circuit, a decoding circuit, an antenna, and the like. A communication I/F according to another standard may be provided.

Examples of near field wireless communication are Bluetooth (registered trademark), IrDA (Infrared Data Association, registered trademark, Zigbee (registered trademark), HomeRF (Home Wireless Frequency, registered trademark), or communication based on Wi-Fi (registered trademark) standard, and the like. Alternatively, for example, communication using an electronic tag may be used.

The telephone network communication I/F 162 may use long distance wireless communication such as W-CDMA (Wideband Code Division Multiple Access, registered mark) or GSM (Global System for Mobile Communications). Although not illustrated, the communication I/F 160 may use other approaches such as optical communication or communication by sound waves, as a means of wireless communication.

For high-definition video or the like which makes the amount of data to be processed greatly increase, using a high-speed and large-capacity communication network such as 5G (5th Generation mobile communication system) or local 5G for wireless communication enables remarkable improvement in the usability.

The HMD 100 further includes a timer 173 as a clock of the main processor 101. In addition, the HMD 100 may include a (LED) lamp 171 to alert the wearer 109. Furthermore, the HMD 100 may include an extended I/F 172.

The extended I/F 172 is a group of interfaces for expanding the functions of the HMD 100. In the present embodiment, the extended I/F 172 includes a charge terminal, a video-and-audio interface, a USB (Universal Serial Bus) interface, a memory interface, or the like. The video-and-audio interface performs input of a video-signal and audio-signal from an external video-and-audio output device, output of a video-signal and audio-signal to an external video-and-audio input device, and the like. The USB interface is provided for connecting a USB device. The memory interface is provided for connecting a memory card and other memory media to transmit and receive data.

The HMD 100 may further include a vibrator for generating a vibration under the control of the main processor 101. The vibrator converts notification data to the wearer 109 transmitted from the HMD 100 into a vibration.

Note that the example of the hardware configuration illustrated in FIG. 3 includes a large number of elements that are not necessarily essential to the present embodiment, and in the configuration without including all these elements, the effects of the present embodiment are not impaired.

### [Functional Blocks]

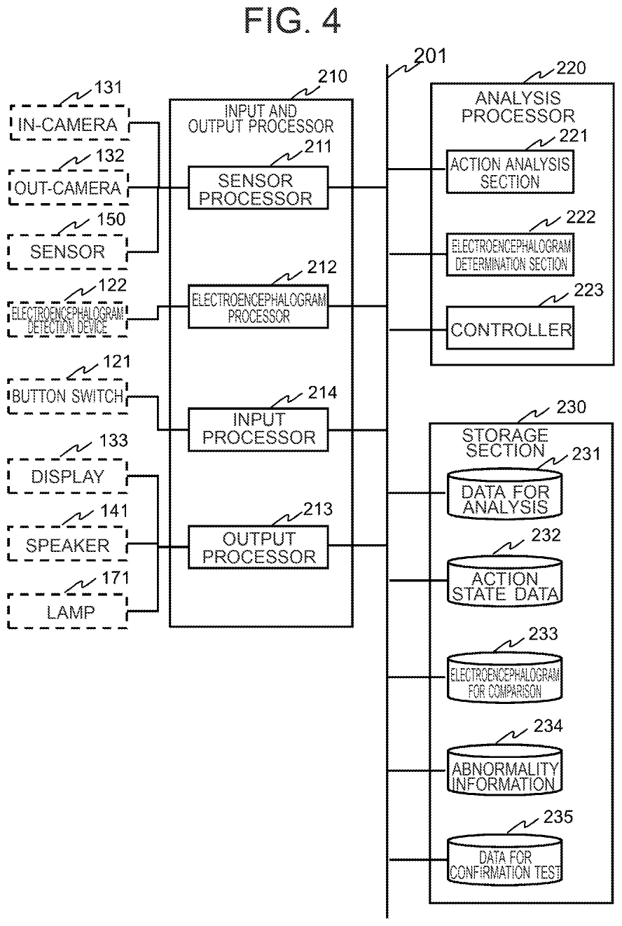

Next, a functional configuration of the information processor of the HMD 100 according to the present embodiment will be described. FIG. 4 is a functional block diagram of the functions of the HMD 100 according to the present embodiment. As illustrated in FIG. 4, the HMD 100 includes an input and output processor 210 and an analysis processor 220. The input and output processor 210 includes a sensor processor 211, an electroencephalogram processor 212, an output processor 213, and an input processor 214. The analysis processor 220 includes an action analysis section (action analysis means) 221, an electroencephalogram determination section (electroencephalogram determination means) 222, and a controller (control means) 223. These sections are connected to each other via a bus 201 to transmit and receive data to and from each other.

The HMD 100 also includes a storage section 230 that stores various types of data used for processing by the analysis processor 220 or obtained as a result of the processing. The storage section 230 stores data for analysis 231, action state data 232, an electroencephalogram for comparison 233, abnormality information 234, and data for confirmation test 235. The storage section 230 is provided in the memory and storage 110.

The sensor processor 211 processes the signals detected by the various sensors of the sensor 150 and the imaging signals received from the in-camera 131 and the out-camera 132. In the present embodiment, the sensor processor 211 brings these signals into a state (action data) allowing to be processed by the action analysis section 221, and outputs the state (action data) to the action analysis section 221. In addition, the sensor processor 211 outputs a sensor signal to the controller 223 as necessary. An action measurement section (action measurement device) is configured with the sensor 150 and the sensor processor 211.

Note that the sensor processor 211 also carries out the general input interface processing for the HMD 100, for example, by analyzing images acquired from the in-camera 131 and the out-camera 132 to receive a gesture input made by the wearer 109 and determining an intention of the wearer 109 based on a signal detected by the sensor 150.

The electroencephalogram processor 212 brings the electroencephalogram detected by the electroencephalogram detection device 122 into a state (electroencephalogram data) allowing to be processed by the electroencephalogram determination section 222, and outputs it to the electroencephalogram determination section 222. Note that an electroencephalogram measurement section (electroencephalogram measurement device) is configured with the electrodes, the electroencephalogram detection device 122, and the electroencephalogram processor 212. The electroencephalogram processor 212 also outputs the electroencephalogram data to the controller 223 as necessary.

The output processor 213 outputs the result of processing by the analysis processor 220 to the output device. In the present embodiment, the output processor 213 outputs the result to, for example, the display 133, the speaker 141, the lamp 171, and the like.

The input processor 214 processes the data input via the input I/F 120. In the present embodiment, the input processor 214 detects, for example, that the button switch 121 has been pressed. The input processor 214 outputs the result of processing to the analysis processor 220.

The action analysis section 221 carries out the action analysis processing of analyzing whether the wearer 109 is in an awake state (hereinafter, referred to as “awake”) or not (hereinafter, referred to as “sleep”) based on the action data received from the sensor processor 211. The action analysis section 221 analyzes the state of the wearer 109 using the signals from the various sensors of the sensor 150 and the data for analysis 231 held in advance.

The action analysis section 221 identifies, for example, the posture, state of motion, opening and closing of the eyes, and the like of the wearer 109 based on the sensor signals, and determines whether the wearer 109 is awake or sleeping based on the result of identification. The action analysis section 221 outputs the result of analysis to the electroencephalogram determination section 222 as an analysis result signal, and stores the result of analysis in the storage section 230 as the action state data 232 in association with the time of analysis. The action analysis section 221 receives the action data at predetermined time intervals and carries out the action analysis. The time interval is, for example, minutes per unit.

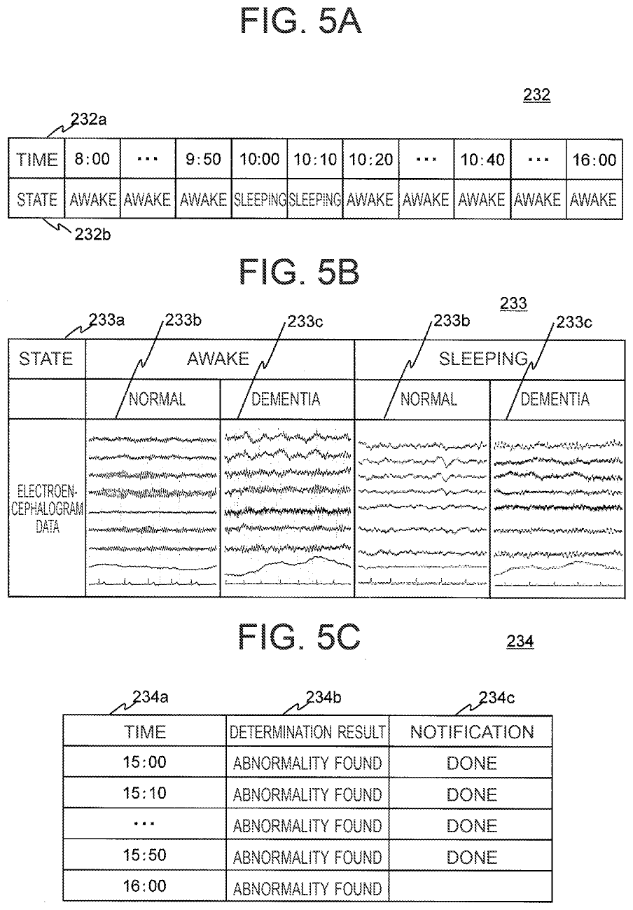

FIG. 5A illustrates an example of the action state data 232. Here, the example is for the case in which the action analysis section 221 receives the action data every ten minutes and carries out the action analysis processing. The state 232b obtained from the result of analysis is registered in association with the start time (time 232a) of the action analysis. In the example of FIG. 5A, the action analysis is carried out from 8:00 to 16:00, and the wearer 109 was sleeping from 10:00 to just before 10:20 while he or she was awake during the other periods of time.

The posture of the wearer 109 is determined using, for example, the surrounding image acquired by the out-camera 132 and the outputs from the acceleration sensor 154, the gyro sensor 155, and the like. For example, if the predetermined change does not appear in the outputs from the acceleration sensor 154 or the gyro sensor within a predetermined time or more, the action analysis section 221 determines that the wearer 109 is sleeping. When these outputs indicate the state in which the wearer 109 is standing or sitting, the action analysis section 221 determines that the wearer 109 is awake. When the outputs indicate that the wearer 109 is lying down, the action analysis section 221 determines that the wearer 109 is sleeping. The state of motion of the wearer 109 is determined based on, for example, an image of the surroundings acquired by the out-camera 132 and the outputs from the acceleration sensor 154, the gyro sensor 155, and the like. When the motion of the wearer 109 is found, the action analysis section 221 determines that the wearer 109 is awake. For determining the opening and closing of the eyes, the action analysis section 221 analyzes an image obtained by the in-camera 131 in which the eyes of the wearer 109 are captured. When the result of analysis indicates that the eyes are open, the action analysis section 221 determines that the wearer 109 is awake, while, when it indicates that the eyes are closed, the action analysis section 221 determines that the wearer 109 is sleeping. The various criteria for this determination are stored in the data for analysis 231.

The action analysis section 221 not only determines whether the wearer 109 is awake or sleeping, but also may divide the action state of the wearer 109 further in more detail for determination. For example, the action state during awake may be more divided into a resting state, a state other than the resting state, or the like. The state other than the resting state includes, for example, the state in which the wearer 109 is moving such as walking or running, having meal, and the like.

The electroencephalogram determination section 222 determines whether an abnormality is found in the electroencephalogram of the wearer 109 based on the electroencephalogram data processed by the electroencephalogram processor 212 and the result of analysis received from the action analysis section 221. In the present embodiment, the electroencephalogram determination section 222 determines whether a suspicion of dementia is found in the electroencephalogram of the wearer 109. The electroencephalogram determination section 222 outputs the result of determination to the controller 223 as a determination result signal. Furthermore, when determining “an abnormality is found”, the electroencephalogram determination section 222 records the abnormality information 234 in the storage section 230.

The electroencephalogram determination section 222 compares the electroencephalogram data as received with the electroencephalogram for comparison 233, which was stored in the storage section 230 in advance, to determine whether there is an abnormality.

FIG. 5B illustrates an example of the electroencephalogram for comparison 233 stored in the storage section 230. In general, a state of an electroencephalogram varies depending on action states (waking or sleeping). As the electroencephalogram for comparison 233, an electroencephalogram 233b in the normal state and an electroencephalogram 233c in the abnormal state are stored in association with action states 233a, respectively. In the example illustrated in FIG. 5B, for during awake and during sleep, normal (healthy) electroencephalogram data and dementia electroencephalogram data are stored as the electroencephalograms for comparison 233, respectively. The electroencephalogram determination section 222 sets, as the result of determination, the one from among the normal electroencephalogram data and the dementia electroencephalogram data which matches the electroencephalogram as obtained more than the other.

Note that each electroencephalogram data stored as the electroencephalogram for comparison 233 is selected and stored in advance from general case data or the like, which matches the attribute information about the wearer 109. The attribute information includes, for example, age, gender, and the like. The electroencephalogram data may be selected and stored in consideration of a medical history or the like.

For making determination about the action state of the wearer 109 during awake further in detail, the electroencephalogram 233b in the normal state and the electroencephalogram 233c in the abnormal state may be stored, as the electroencephalogram for comparison 233, in association with each action state during awake (for example, resting state, moving state, eating state, or the like).

When the electroencephalogram determination section 222 determines “an abnormality is found” in the electroencephalogram of the wearer 109, the controller 223 carries out predetermined processing. In the present embodiment, upon receiving a determination result signal indicating an abnormality has been found from the electroencephalogram determination section 222, the controller 223 records the data (abnormality information 234) showing “an abnormality is found” in the storage section 230.

Upon receiving a determination result signal indicating “no abnormality is found”, the controller 223 determines whether the abnormality information 234 is stored in the storage section 230. If the abnormality information 234 is stored, the controller 223 generates the output information and transmits it to the output processor 213, and deletes the abnormality information 234 that had not been notified.

Note that the controller 223 may be configured to record the abnormality information in the storage section 230 in association with the time and set a notification completion flag when generating the output information. In this case, upon receiving the determination result signal indicating “no abnormality is found”, the controller 223 determines whether the abnormality information that has not been notified is recorded. If the abnormality information that has not been notified is found, the controller 223 generates the output information.

FIG. 5C illustrates an example of the abnormality information 234 in this case. Determination results 234b showing “an abnormality is found” are recorded in association with times 234a, respectively. In addition, notification columns 234c showing whether abnormality information has been notified are also provided. Recording the determination result 234b in association with the time and recording whether notification has been made allows a record in which determination of “an abnormality is found” has been made to be kept.

The output information to be transmitted by the controller 223 includes, for example, a message to be displayed on the display 133. It may also include the audio data to be output from the speaker 141 and a lighting signal of the lamp 171.

The controller 223 may be further configured to carry out the confirmation processing after transmitting the output information to the output processor 213 and before setting the record as already being notified. In the confirmation processing, a predetermined testing (test for confirmation) is provided to the wearer 109 to confirm whether the notification is reliably recognized by the wearer 109.

For example, the controller 223 causes the wearer 109 to perform a predetermined operation in the confirmation test. The operation to be performed by the wearer 109 is, for example, inputting a password, operating a predetermined operation unit, making a gesture, answering a predetermined question, calculating, identifying an image, and the like. The controller 223 causes the wearer 109 to perform one or a combination of these. An execution instruction and model answers are stored in advance in the storage section 230 as the data for confirmation test 235.

The controller 223 causes the output processor 213 to output the execution instruction, and accepts an operation made by the wearer 109 as an answer via the input processor 214 or the sensor processor 211. Any method may be employed for presenting the execution instruction and inputting the answer as long as it can be used for the HMD 100.

For example, for inputting a password as the execution instruction, a password input screen is displayed on the display 133 so that the wearer 109 can input the password therethrough. The input of the password is detected based on the line-of-sight direction of the wearer 109. For example, it is detected by determining whether the line-of-sight direction of the wearer 109 designates each key. Alternatively, the wearer 109 can input the password via the button switch 121. The wearer 109 may speak the password to allow the microphone 142 to detect the password. If displaying a VR (Virtual Reality) keyboard, the touch of the wearer 109 may be detected by the image analysis and the range sensor 153.

In identifying an image, for example, an image of a vegetable, a fruit, an animal, or the like is displayed so that the wearer 109 can input the name thereof. In the same manner as the case of a password, the wearer 109 can input the name by a line-of-sight input on a keyboard as displayed, by audio input, or virtual input.

If providing a calculation, a calculation problem is displayed on the display 133 so that the wearer 109 can input the answer via a keyboard that appears on the display 133, or by audio input or virtual input.

Alternatively, for example, a stimulus of light may be provided to determine whether the brain responds thereto. In this case, as the stimulus of light, for example, repeatedly the whole image or a part of the image that appears on the display may be displayed or hidden at regular intervals, bright images, characters, and the like similar to the lamps, which are the image for confirmation of reaction may be displayed, the image being displayed may be displayed or hidden, the lamp 171 may be turned on and blinked, and the like. The determination is made based on whether a specific change in the electroencephalogram is found at the timing of providing the stimulation of light. The electroencephalogram processor 212 processes the electroencephalogram detected by the electroencephalogram detection device 122, and the controller 223 checks it.

When receiving the correct answer, the controller 223 determines that the wearer 109 is in a state allowing the notification to be recognized and thus the notification has been reliably transmitted to the wearer 109, and then carries out the processing of erasing the abnormality information. On the other hand, if not receiving the correct answer, the controller 223 carries out the notification processing again.

### [Output Processing]

In the following, an example of output by the output processor 213 will be described. Upon receiving the output information from the controller 223, the output processor 213 outputs an abnormality notification for notifying that an abnormality has been found through an output device corresponding to the output information.

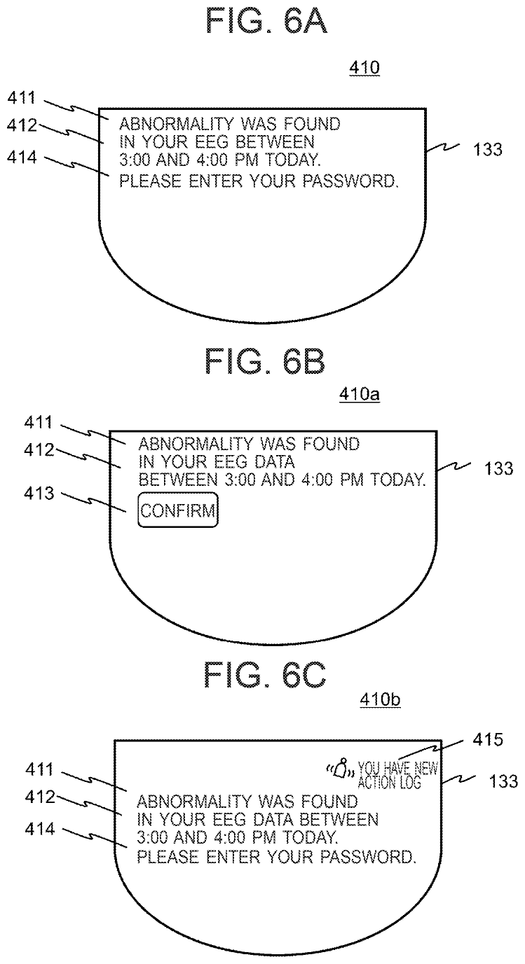

FIG. 6A illustrates a display example 410 on the display 133 in the case of outputting the notification by displaying it. In this case, the output processor 213 displays a time 411 at which the abnormality in the electroencephalogram is detected, a message 412, and a message 414 for confirmation test. Here, for example, the message 414 for confirmation test is provided to cause the wearer 109 to input a password. Upon accepting the input of the password from the wearer 109, the input processor 214 notifies the controller 223 of the reception of the password. The controller 223 determines whether the password as input is valid, and deletes the abnormality information 234 if it is valid.

In the case of outputting the notification by means of audio data, the output processor 213 generates an audio message and outputs it through the speaker 141. In addition, the lamp 171 may be flashed or turned on so as to cause the wearer 109 to be aware of the notification. In the case of the HMD 100 having a vibration function, the vibration function may be activated for providing the notification. A message for advising the wearer 109 to go to the hospital may be added.

### [Techniques for Acquiring Electroencephalogram]

Next, a method of acquiring an electroencephalogram will be briefly described. As the techniques for acquiring an electroencephalogram, for example, referential recording, bipolar recording, average potential reference electrode, source derivation have been known.

The referential recording (unipolar lead) is the method of acquiring (recording) a potential difference between two points which are a probe electrode on the scalp and a reference electrode placed on the ear lobe as an electroencephalogram. This method allows the electric potential distribution of the whole brain to be easily known, and is generally used for determining a fundamental wave and applying an activation method.

The bipolar recording is the method of recording a potential difference between two probe electrodes on the scalp, and suitable for searching the focal point of the localized abnormal wave.

The average potential reference electrode is the method of recording an electroencephalogram based on an average value of potentials obtained when a predetermined resistance is applied to each electrode on the scalp. According to this method, the absolute value at each electrode is not known, however, it allows a relative difference, that is, a state of a relative distribution to be detected.

The source derivation is the method of recording an electroencephalogram based on a weighted average potential of electrodes surrounding around an electrode to be introduced. In this method, spreading potential components are cancelled, and thus only a component immediately below the probe electrode to be introduced can be detected with the improved SN ratio.

Any of the methods above may be used in the present embodiment. Depending on the technique to be used, electrodes are placed on the support portion 190 or the main body portion 180. For example, if using the technique of referential recording, electrodes are arranged in portions corresponding to the earlobe and the forehead. An electrode placed on the earlobe is used as a reference electrode.

### [Analysis and Evaluation Processing]

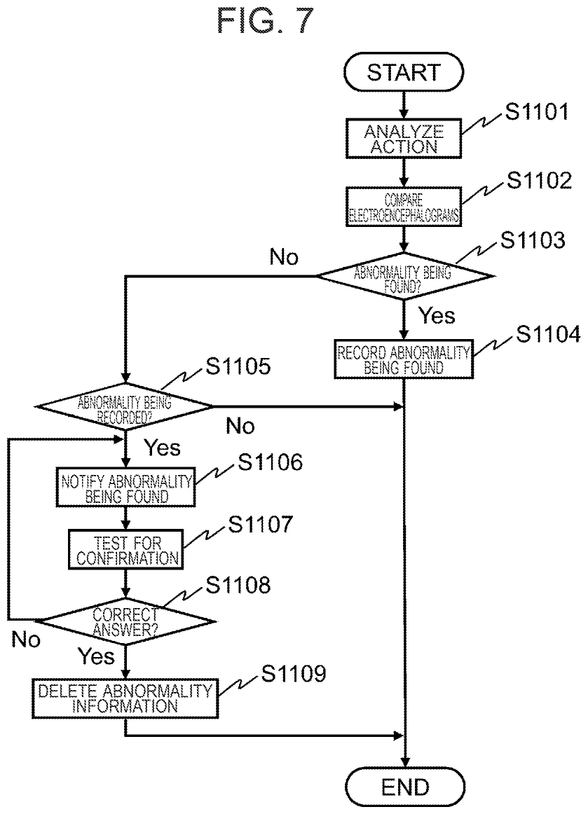

In the following, a flow of an analysis and evaluation processing by the HMD 100 according to the present embodiment will be described. FIG. 7 is a processing flow of the analysis and evaluation processing according to the present embodiment. For example, the HMD 100 executes the following analysis and evaluation processing at predetermined intervals. The predetermined time intervals are, for example, intervals of a few minutes.

Firstly, the action analysis section 221 carries out the action analysis processing of analyzing the state of the wearer 109 based on the sensor signal processed by the sensor processor 211 (step S1101). Here, the action analysis section 221 identifies whether the wearer 109 is awake or sleeping. The action analysis section 221 outputs the result of analysis to the electroencephalogram determination section 222. The result of analysis is recorded in the storage section 230 as the action state data 232 in association with the time.

The electroencephalogram determination section 222 acquires an electroencephalogram obtained in a predetermined period of time (electroencephalogram determination period) and compares it with the electroencephalogram for comparison 233 (step S1102). The electroencephalogram determination section 222 identifies the electroencephalogram data having the highest degree of matching from among the electroencephalograms for comparison 233 to determine whether there is an abnormality in the electroencephalogram of the wearer 109 (step S1103). In other words, when the electroencephalogram data in the normal state has the highest degree of matching, it is determined as being normal, and when the electroencephalogram data in the dementia state has the highest degree of matching, it is determined as having dementia (abnormal). When comparing the data, the electroencephalogram determination section 222 uses the result of analysis by the action analysis section 221. The electroencephalogram determination section 222 outputs a determination result signal indicating the result of determination to the controller 223.

When the electroencephalogram determination section 222 determines “an abnormality is found”, the controller 223 records the abnormality information 234 in the storage section 230 (step S1104).

On the other hand, if the electroencephalogram determination section 222 does not determine “an abnormality is found”, the controller 223 determines whether the abnormal information 234 has been recorded in the storage section 230 (step S1105). If it has not been recorded, the controller 223 ends the processing as it is.

If the abnormality information 234 has been recorded, the controller 223 generates the output information and carries out the abnormality notification processing of notifying the wearer 109 of the abnormality being found (step S1106). Then, the controller 223 provides the test for confirmation (step S1107).

The controller 223 repeatedly carries out the abnormality notification processing and the test for confirmation until the correct answer of the test for confirmation is obtained (step S1108). In the present embodiment, for example, when a valid input operation is made within a predetermined period of time, the controller 223 determines that the correct answer has been obtained.

Upon obtaining the correct answer, the controller 223 deletes the abnormality information (step S1109), and ends the processing.

As described above, the HMD 100 according to the present embodiment includes an electroencephalogram measurement device (electroencephalogram detection device 122 and electroencephalogram processor 212) configured to output electroencephalogram data on the wearer 109 who is a user, an action measurement device (sensor 150, in-camera 131, out-camera 132, and sensor processor 211) configured to output action data on the wearer 109, and the controller 223 configured to determine whether an abnormality is found in the electroencephalogram data based on the electroencephalogram data and the action data, and carries out the predetermined assistance processing for the wearer 109 when the result of determination indicates that the abnormality has been found.

In the present embodiment, whether an abnormality is found in the electroencephalogram is determined by taking into consideration not only the electroencephalogram data on the wearer 109 but also the action data on the wearer 109, and if an abnormality has been found, the controller 223 carries out the assistance processing for the wearer 109.

The HMD 100 according to the present embodiment further includes the action analysis section 221 configured to determine whether the wearer 109 is awake based on the action data, and the storage section 230. The controller 223 stores the abnormality information 234 in the storage section 230 in response to the result of determination indicating “an abnormality is found”. The controller 223 provides an abnormality notification for notifying the wearer 109 of the abnormality being found in the electroencephalogram if the abnormality information is stored in the storage section 230 in the case where the result of determination indicates “no abnormality is found” and also the result of determination by the action analysis section 221 indicates the wearer 109 is awake. The controller 223 deletes the abnormality information 234 after providing the abnormality notification.

The HMD 100 according to the present embodiment with the configuration as described above notifies the wearer 109 of suspicion of an abnormality such as dementia while he or she is in the normal state if it appears in the electroencephalogram. In the early stages of dementia, the dementia state and the normal state tend to occur alternately. The HMD 100 according to the present embodiment analyzes the electroencephalogram of the wearer 109 and upon detecting a dementia state, notifies the wearer 109 of a suspicion of dementia while he or she is in the normal state (while the degree of suspicion of dementia is low and the consciousness of the wearer 109 is clear). This enables assistance for causing the wearer 109 to be aware of an abnormality such as dementia at an early stage. In this assistance, the HMD 100 may advise the user to go to the hospital. Thus, the wearer 109 is encouraged to take early examinations and treatment, which increases the possibilities that the wearer 109 would go to the hospital at the early stage and take diagnosis and treatment. As a result, it is possible to prevent the symptoms from progressing while the user himself or herself has not been aware of the abnormality, and to delay the progression of the disease such as dementia as much as possible.

Furthermore, the HMD 100 according to the present embodiment is configured to, in case that there is a suspicion of an abnormality, repeatedly carry out tests for confirmation and provides notifications until a correct answer is obtained. This enables a notification to be provided reliably while the wearer 109 can make judgement normally.

### <First Modification>

In the embodiment described above, the controller 223 provides the test for confirmation after notifying the wearer 109 of the abnormality being found, however, the test for confirmation is not necessarily provided. In other words, when the electroencephalogram determination section 222 determines “an abnormality is found”, the controller 223 may notify the wearer 109 of the abnormality being found without providing the test for confirmation at the timing when it is determined “no abnormality is found”, and only receive a confirmation operation from the wearer 109. FIG. 6B illustrates a display example 410a on the display 133.

In this example, the output processor 213 displays the time 411 at which the abnormality was detected in the electroencephalogram and the message 412. In addition, the output processor 213 provides, for example, a confirmation button display 413 to ensure that the wearer 109 has received the message. Upon accepting an operation from the wearer 109, the input processor 214 outputs it to the controller 223. Upon receiving that the operation with respect to the confirmation button display 413 has been made, the controller 223 deletes the abnormality information 234.

Note that the confirmation button display 413 may be provided.

The present modification enables further reduction in the burden on the wearer 109, and also can realize early detection of an abnormality in the brain.

### <Second Modification>

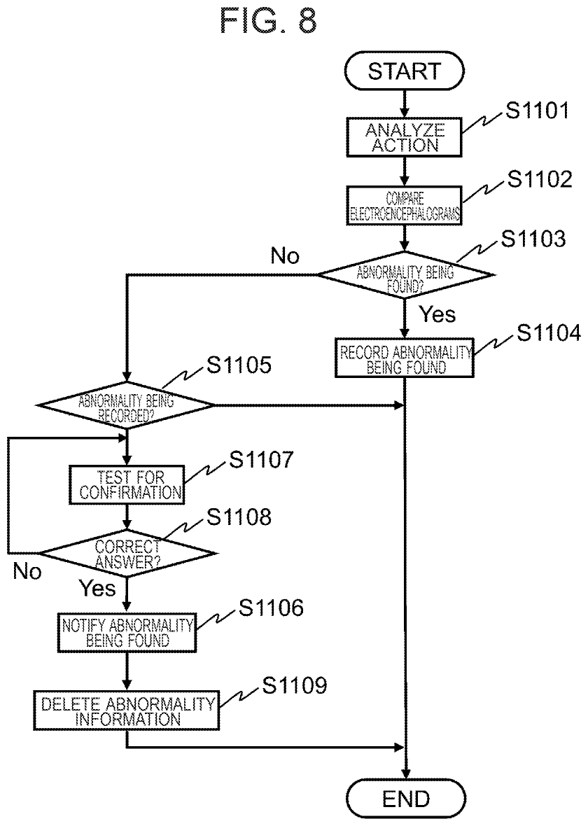

Furthermore, in the embodiment described above, the controller 223 carries out the abnormality notification processing and then provides the test for confirmation, however, the order of execution of them may be reversed. For example, as illustrated in FIG. 8, if the abnormality information has been recorded in step S1105, the controller 223 firstly may provide the test for confirmation (step S1107), and then, upon receiving the correct answer (step S1108), provide the notification of the abnormality being found (step S1106).

According to the present modification, the notification does not have to be repeatedly provided many times, which results in reduction in the troublesome.

### <Third Modification>

Still further, in the embodiment described above, typical waveforms are registered as the electroencephalogram data for comparison, however, the present invention is not limited thereto. For example, as a normal waveform in an electroencephalogram, the waveform in the electroencephalogram of the wearer 109 in his or her normal state may be acquired in advance and registered.

This improves the accuracy in determination.

The electroencephalogram determination section 222 may analyze the shape of the electroencephalogram data to determine whether an abnormality is found, instead of comparing the electroencephalogram data with the pre-registered data. In other words, the electroencephalogram determination section 222 determines that there is a suspicion of dementia when the electroencephalogram data specific to the abnormal state (in the present embodiment, dementia) is obtained. For example, when a slow wave (for example, 8 wave having a long wavelength) appears even while the wearer 109 is awake, it is determined that there is a suspicion of dementia.

### <Fourth Modification>

Still further, the HMD 100 may be configured to transmit a report to a predetermined notification destination when an abnormality occurs in the wearer 109 immediately after detecting an impact.

When detecting a large impact, the action analysis section 221 provides the electroencephalogram determination section 222 with a report that the impact was detected together with a result of analysis on whether the wearer 109 is awake or sleeping. When determining “an abnormality is found” immediately after detecting the impact, or when the electroencephalogram shows a predetermined state, the electroencephalogram determination section 222 transmits an abnormality occurrence signal to the controller 223.

The predetermined state of the electroencephalogram, by which it would be determined that the abnormality occurrence signal should be transmitted is, for example, the case in which an abnormality is detected by analyzing a signal acquired by the biometric information acquisition sensor 156, which shows an amplitude larger than a predetermined threshold value appeared in the electroencephalogram compared to the one obtained before the impact is detected.

Upon receiving the abnormality occurrence signal, the controller 223 transmits a report to a predetermined notification destination. The destination to be reported is, for example, a relative, the police, a hospital, a security company, or the like which is registered in advance in the storage section 230. The report is provided by, for example, transmitting an e-mail to the address of the notification destination via the communication I/F 160.

Note that transmission of a report is not limited for the case in which an impact is detected. For example, in case that a valid input operation has not been obtained even after the tests for confirmation were repeatedly provided for a predetermined number of times, the controller 223 may record this fact and transmit a report to the outside. In this case, the notification destination is, for example, a relative, a protector, a guardian, a hospital, an insurance company, or the like which is set in advance.

In addition, in case that determination of “an abnormality is found” has been made for a predetermined period of time or more, the controller 223 may also transmit a report thereof to the outside. In this case, the notification destination is, as described above, a family, a protector, a hospital, a guardian, an insurance company, or the like which is set in advance.

### <Fifth Modification>

The HMD 100 may be the one of separate type in which the display 133 and the function unit implemented by the main processor 101 are separately provided. In this case, the display 133 only includes a display control function and a wireless or wired communication I/F, and communicates with each function unit to transmit and receive signals and data with each other.

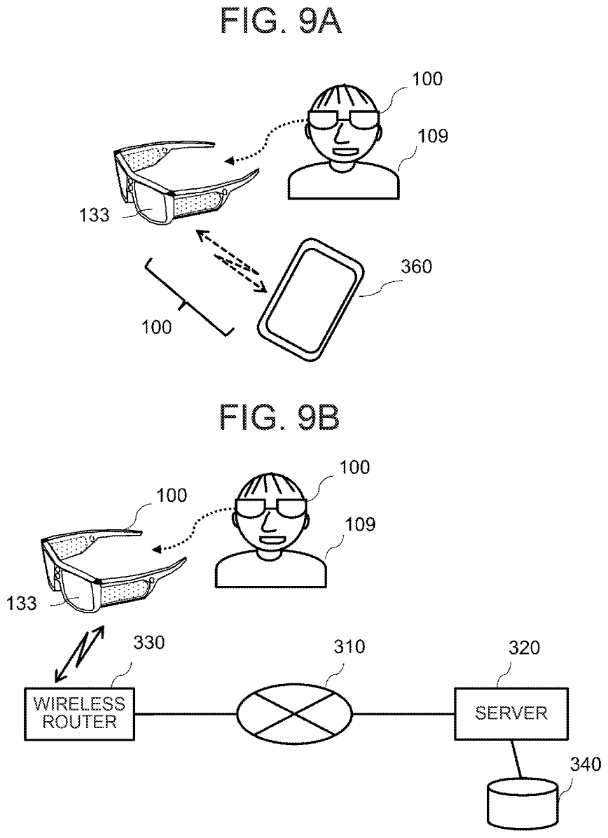

For example, as illustrated in FIG. 9A, each function unit may be implemented by an external device such as a smartphone 360 or a mobile terminal device. The display 133 transmits and receives signals and data to and from each function unit in the mobile terminal via a wireless communication I/F. In this case as well, transmission and reception may be performed via a wired cable. Note that each function unit may be implemented by a PC (Personal Computer) if the wearer 109 is in the home.

As illustrated in FIG. 9B, the external device that implements some functions may be a server 320 connected via a network 310.

The HMD 100 outputs the sensor data and electroencephalogram data as acquired to the external device (server 320) via the wireless communication I/F 161. The wireless communication I/F 161 is connected to the network 310 via a wireless router 330.

Upon acquiring the sensor signal to be used to determine the state of the wearer 109, the HMD 100 transmits it to the server 320 via the wireless communication I/F 161. In the same manner, upon acquiring the electroencephalogram data, the HMD 100 transmits it to the server 320 via the wireless communication I/F 161.

On the side of the server 320, the sensor signal and electroencephalogram data are managed for each transmission source. The signal and data are controlled, for example, in a storage device 340 accessible by the server 320. The server 320 carries out the analysis and evaluation processing as described above for each transmission source at predetermined time intervals, and notifies the transmission source of the result of analysis.

Note that, among the processing relating to the electroencephalogram analysis according to the present embodiment, the ones to be carried out by an external device such as the server 320 and the other ones to be carried out by the HMD 100 can be arbitrarily decided.

For example, the server 320 may cover the functions of the action analysis section 221 and the electroencephalogram determination section 222 while the HMD 100 executes the functions of the controller 223. In this case, the server 320 determines whether an abnormality is found in an electroencephalogram at predetermined time intervals, and notifies the HMD 100 of the result of determination for each determination. The HMD 100 records the abnormality information 234 in the storage section 230 if receiving the result of determination of “an abnormality is found”, and then notifies the wearer 109 of “an abnormality is found” at the timing when receiving the result of determination of “no abnormality is found”.

The server 320 may be provided with all the functions of the analysis processor 220. In this case, the server 320 transmits the output information to the HMD 100. When the HMD 100 receives the output information from the server 320, the output processor 213 carries out the output processing to notify the wearer 109 of “an abnormality is found” at that timing.

Note that the output information to be transmitted from the server 320 may be, for example, a message for notifying an abnormality in an electroencephalogram, or may be a signal indicating an abnormality has been found. In the case of the latter, the HMD 100 generates and outputs a predetermined message.

Note that the electroencephalogram for comparison 233 to be used in the server 320 is not limited to the one stored for each state (awake, sleeping). For example, the information may be classified and stored according to attribute information such as age, gender, and medical history. In this case, the attribute information about the wearer 109 is registered in the server 320 in advance, and the server 320 selects the electroencephalogram for comparison 233 to be used based on the registration information when making determination.

In the same manner as the modification described above, the electroencephalogram data on the wearer 109 in the normal state may be registered in the server 320 as the electroencephalogram for comparison 233.

Note that, as the action analysis device having the function of the action analysis section 221, the electroencephalogram determination device having the function of the electroencephalogram determination section 222, and the controller having the function of the controller 223, the functions provided in the HMD 100 according to the present embodiment may be implemented by separate devices, respectively, and a user assistance system that carries out the same processing as that of the HMD 100 according to the present embodiment may be configured as a whole.

### <Sixth Modification>

Still further, in the embodiment and modifications described above, whether an abnormality is found in the electroencephalogram of the wearer 109 is determined at predetermined time intervals, however, the timing of making determination is not limited thereto. For example, as the batch processing, it may be determined after the data of one day is collected. The abnormality information is recorded if there is a period of time showing “an abnormality is found”. Then, on the next day, the notification processing is carried out at the timing when the wearer 109 has worn the HMD 100. In this case, the notification is not necessarily provided to the wearer who is in the normal state, and thus the test for confirmation needs to be provided or the “confirmation” button needs to be displayed.

### Second Embodiment

Next, a second embodiment of the present invention will be described. In the first embodiment, when an abnormality is found in the electroencephalogram, for example, when there is a suspicion of dementia, the wearer 109 himself or herself is notified of the abnormality. In the present embodiment, when an abnormality is found in the electroencephalogram, a function of a predetermined application program (hereinafter, simply referred to as an application) mounted on the HMD 100 is restricted.

In the present embodiment, when an abnormality is found in the electroencephalogram, the HMD 100 determines that the wearer 109 is not in the condition in which he or she can make conscious judgement, and prevents the wearer 109 from operating the function that is assumed to require a restriction. For example, the HMD 100 hides an icon of an application for financial transaction or shopping which involves payment or the like on the menu screen. Alternatively, for example, the function of payment may be restricted by individual setting of the function of the application.

Hereinafter, the present embodiment will be described focusing on the features different from those of the first embodiment.

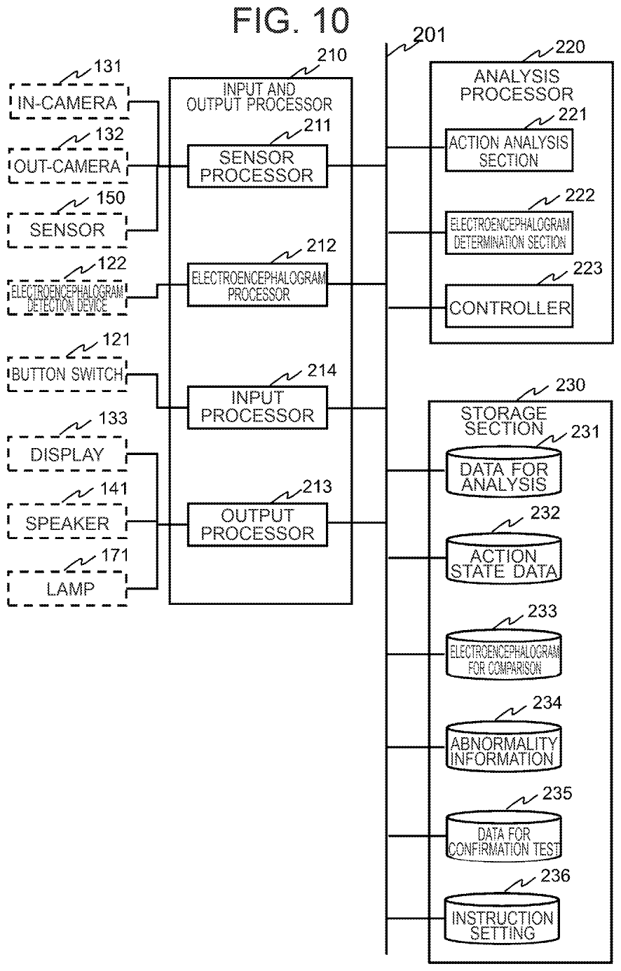

The appearance and hardware configuration of the HMD 100 according to the present embodiment are the same as those of the HMD 100 according to the first embodiment. The functional configuration according to the present embodiment is also the same as that according to the first embodiment. However, differences can be found in the processing by the controller 223 according to the present embodiment, and also, as illustrated in FIG. 10, in an instruction setting 236, which will be described later, stored in the storage section 230 in advance for the processing by the controller 223.

When the electroencephalogram determination section 222 determines “an abnormality is found”, the controller 223 records the abnormality information in the same manner as the first embodiment. Furthermore, in the present embodiment, the controller 223 carries out the processing (app setting processing) for restricting the operation of the application. Note that, in the appl setting processing, when the electroencephalogram determination section 222 determines “no abnormality is found”, the controller 223 carries out the processing of canceling the restriction that was set based on the determination of “an abnormality is found”.

Specifically, the controller 223 outputs a control signal (restriction signal) to various applications installed in the HMD 100 in accordance with the predetermined instruction setting 236. In the present embodiment, the controller 223 outputs, for example, a startup stop signal for stopping the startup and a setting change signal for changing the setting of the application to each application in accordance with the instruction setting 236.

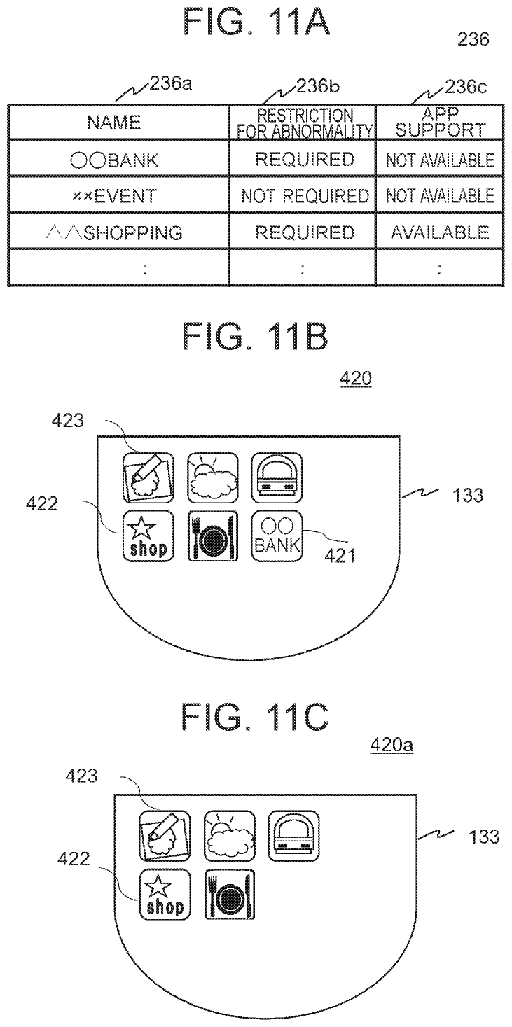

FIG. 11A illustrates an example of the instruction setting 236. In the instruction setting 236, for each application (name) 236a installed in the HMD 100, a restriction for abnormality 236b indicating whether the restriction is required in case that an abnormality is found, and an app support 236c indicating whether the application supports the restriction are registered.

When “required” is registered in the restriction for abnormality 236b and “not available” is registered in the app support 236c, the controller 223 determines that the application needs to be restricted but the application does not have a preparation for restriction. Then, the controller 223 outputs, directly to the application, the startup stop signal for stopping the startup.

When “required” is registered in the restriction for abnormality 236b and “available” is registered in the app support 236c, the controller 223 determines that the application needs to be restricted and a function for restriction is available in the application. Then, the controller 223 outputs a setting change signal to the application.

When “not required” is registered in the restriction for abnormality 236b, the controller 223 determines that the application does not need to be restricted, and does not output a restriction signal to the application.

In the example illustrated in FIG. 11A, for the ◯◯ bank application, “required” is registered as the restriction for abnormality 236b and “not available” is registered as the app support 236c. For the xx event application, “not required” is registered as the restriction for abnormality 236b, and “not available” is registered as the app support 236c. For the ΔΔ shopping application, “required” is registered as the restriction for abnormality 236b and “available” is registered as the app support 236c.

When it is determined “an abnormality is found”, the controller 223 outputs the startup stop signal to the ◯◯ bank application, and outputs the setting change signal to the ΔΔ shopping application. On the other hand, the controller 223 does not output any restriction signal to the xx event.

The application that has received the startup stop signal is not started even if the wearer 109 provides a startup instruction. Note that, for the application that has received the startup stop signal, the icon thereof may be hidden on the menu display.

The application that has received the setting change signal locks a predetermined function so that it cannot be used. For example, in the ΔΔ shopping application, the payment function is locked. As a result, the wearer 109 cannot make a payment although he or she can start the application and view (browse) items.

FIG. 11B and FIG. 11C illustrate a menu display example 420 in the normal case and a menu display example 420a in the case where it is determined “an abnormality is found” (abnormal case), respectively.

As illustrated in FIG. 11B and FIG. 11C, an icon 421 of the bank application that has received the startup stop signal is displayed in the menu display example 420 in the normal case, however, is not displayed in the display example 420a in the abnormal case. On the other hand, an icon 422 of ΔΔ shopping application that has received the setting change signal is displayed on both the menu display example 420 and the menu display example 420a. However, although the ΔΔ shopping application can be started, the restricted function thereof cannot be used. The function to be restricted is, for example, the function for payment. In other words, in the ΔΔ shopping application, the wearer 109 can browse the items but cannot pay for them. Note that an icon 423 of the xx event application that has not received the startup stop signal nor the setting change signal is displayed on both the menu display example 420 and the menu display example 420a, and all the functions thereof can be used as they are.

The controller 223 records the applications to which it has transmitted the startup stop signal and a control instruction signal. Then, when it is determined that the wearer 109 is in the normal state, the controller 223 provides the wearer 109 with the abnormality notification and cancels these settings. Specifically, the controller 223 transmits a startup stop cancellation signal to the application to which it has transmitted the startup stop signal, and transmits a control cancellation signal to the application to which it has transmitted the control instruction signal. The startup stop cancellation signal is the signal for canceling the stop of activation of the application that has been temporarily stopped not to be started, and the control cancellation signal is the signal for canceling the stop of the function that has been stopped in accordance with the control instruction. Both the signals are collectively referred to as cancellation signals.

### [Analysis and Evaluation Processing]

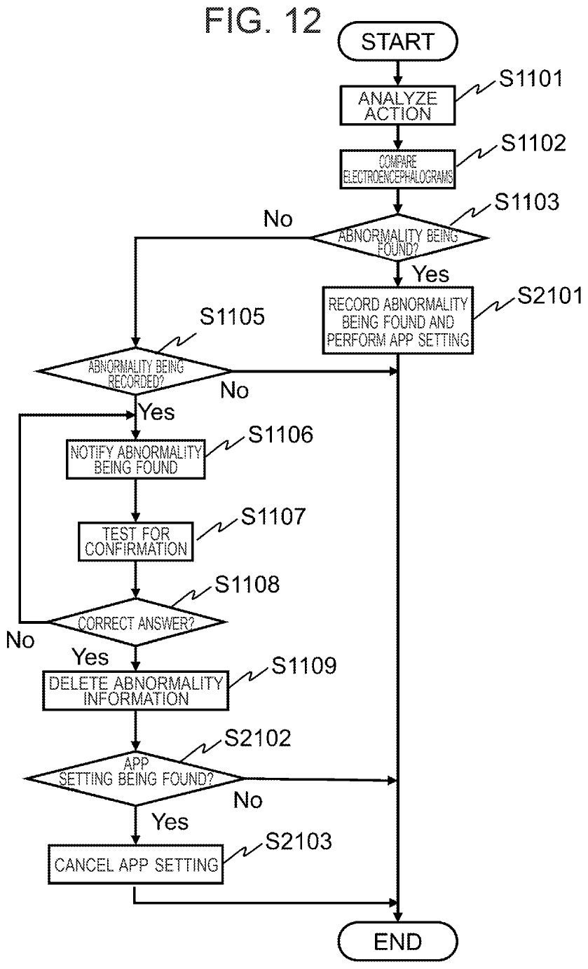

FIG. 12 illustrates a flow of the analysis and evaluation processing according to the present embodiment. This processing is carried out at predetermined time intervals in the same manner as the first embodiment. In the following, in the processing, differences from those of the first embodiment will be mainly described.

In the present embodiment, when it is determined “an abnormality is found”, the controller 223 not only records the abnormality information 234, but also carries out the app setting processing of transmitting a restriction signal to each application installed in the HMD 100 in accordance with the instruction setting 236 (step S2101), and then ends the processing.

On the other hand, when it is determined “no abnormality is found”, the controller 223 firstly determines whether the abnormality information 234 is recorded. If the abnormality information 234 is recorded, in the same manner as the first embodiment, the controller 223 repeatedly provides the abnormality notification processing and the test for confirmation until a correct answer of the test for confirmation is obtained. When the correct answer is obtained, in the same manner as the first embodiment, the controller 223 deletes the abnormality information (step S1109) and determines whether the app setting processing was carried out for any application (step S2102). When finding the application for which the app setting processing was carried out, the controller 223 transmits a cancellation signal to cancel the setting of the application (step S2103), and ends the processing. When not finding any application for which the app setting processing was carried out, the controller 223 ends the processing as it is.

As a result, when the wearer 109 starts the menu screen, the icon of each application is displayed in the manner set at that time.

As described above, in the HMD 100 according to the present embodiment, when the result of determination by the electroencephalogram determination section 222 is “an abnormality is found”, an instruction for restricting the processing is output to a predetermined application.

According to the present embodiment, not only the wearer 109 is notified of an abnormality, but also the function of the HMD 100 is restricted. This can prevent the wearer 109 from carrying out the processing contrary to his or her intention in the state where the abnormality occurs in the electroencephalogram.

### <Seventh Modification>

In the embodiment described above, the instruction setting 236 is registered in advance for each application, and the controller 223 outputs a restriction signal to the corresponding application according to the content of registration. However, the instruction setting 236 may not be registered in advance, and the controller 223 may determine whether to output the restriction signal based on the attribute information or the like about each application, and output the restriction signal as necessary. The controller 223 uses, for example, a setting of a property of an application if available.

### <Eighth Modification>

Note that, in the present embodiment, the application setting processing is carried out immediately after an abnormality is determined, however, the present invention is not limited thereto. Recording of the abnormality information 234 and the app setting processing may be carried out separately.

That is, the controller 223 records the abnormality information 234 and provides a notification in accordance with the processing flow of FIG. 7 and the like, in the same manner as the first embodiment. Then, the controller 223 carries out the app setting processing upon receiving an instruction to display a menu or an instruction to start an application. If the abnormality information 234 is recorded, the controller 223 refers to the instruction setting 236 and carries out the app setting processing described above for the corresponding application. In addition, when the abnormality information 234 is deleted, the controller 223 determines whether the appl setting has been made for any application, and when finding a corresponding application, cancels the setting thereof.

### <Ninth Modification>

Furthermore, in the case of the HMD 100 synchronized with another external device, the setting of the application may also be made synchronized. In the case of the HMD 100 synchronized with one or more external devices, the controller 223 transmits control signals such as a startup stop signal, a control instruction signal, a startup stop cancellation signal, and a control cancellation signal to all the synchronized devices.

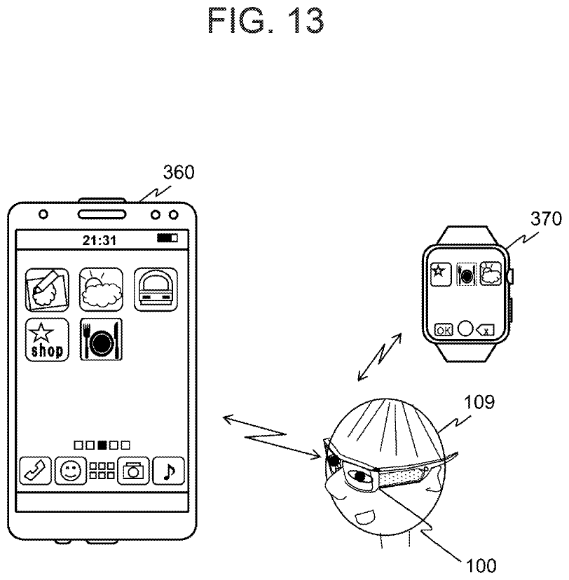

For example, as illustrated in FIG. 13, in the case of the HMD 100 synchronized with a smartphone 360 and a smartwatch 370, the controller 223 outputs each control signal described above to these devices via the communication I/F 160.

Note that the communication with the smartphone 360 and the smartwatch 370 may be carried out by near field wireless communication such as Bluetooth, or by Wi-Fi communication via a wireless router.

The external devices to be synchronized are registered in the HMD 100 in advance.

According to the present modification, when it is determined that an abnormality is found in the electroencephalogram, in the same manner as the HMD 100, the function provided in the registered external device such as the smartphone 360 or the smartwatch 370 can be restricted as well.

When being allowed to be operated by a predetermined guardian, the external device may be configured such that the guardian can cancel the restriction. In this case, the authentication information about the guardian, such as ID and the password is registered in the external device in advance. When an abnormality is determined in the electroencephalogram of the wearer 109 and thus the function of the registered external device is restricted, the guardian inputs the authentication information to the external device, and upon success of the authentication based on the authentication information, the external device can cancel the restriction that has been set in accordance with the instruction from the HMD 100. Note that, in each external device, if the function of which the restriction was cancelled has not been operated for a certain period of time, the restriction may be set again.

### <Tenth Modification>

The HMD 100 does not necessarily restrict the function of the application by its own, but may restrict the function by transmitting a signal to the server that is a service provider of the application. In other words, instead of transmitting a restriction signal such as a startup stop signal or a setting instruction signal to an application in the HMD 100, the HMD 100 transmits a signal (restriction request signal) for requesting a restriction to a server that is a service provider of the application.

In this case, the controller 223 transmits a restriction request signal to the server that is a service provider of the application when the function to monitor the processing by a predetermined application or an application to be monitored carries out the predetermined processing.

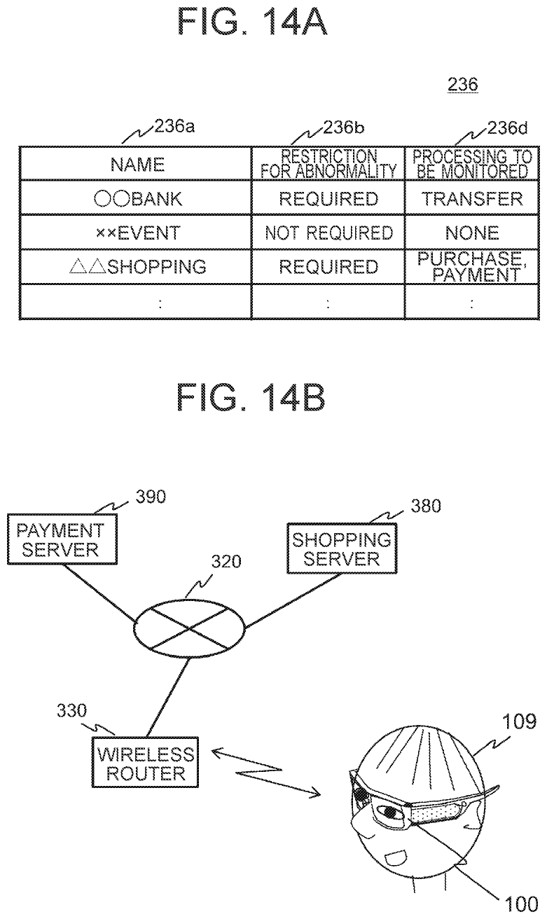

In the present embodiment, in the instruction setting 236, as illustrated in FIG. 14A, processing to be monitored 236d is registered instead of the app support 236c.

While the abnormality information 234 is being recorded, the controller 223 monitors the processing registered in processing to be monitored 236d of an application for which “required” is registered as the restriction for abnormality 236b in the instruction setting 236, for example.

While the abnormality information 234 is being recorded, if the application carries out the processing to be monitored 236d, the controller 223 transmits a restriction request signal to the server that is a service provider of the application. The controller 223 records the server to which it has transmitted the restriction request signal. Then, upon deletion of the abnormality information 234, the controller 223 transmits a restriction cancellation request signal to the server as recorded. The restriction cancellation request signal is the request to cancel the restriction.

In the following, the present modification will be described with a specific example. Here, as illustrated in FIG. 14B, an example in which the wearer 109 uses an application for ΔΔ shopping will be described. A shopping server 380 serving as the service provider of the application and a payment server 390 are connected to the HMD 100 through the network 310.

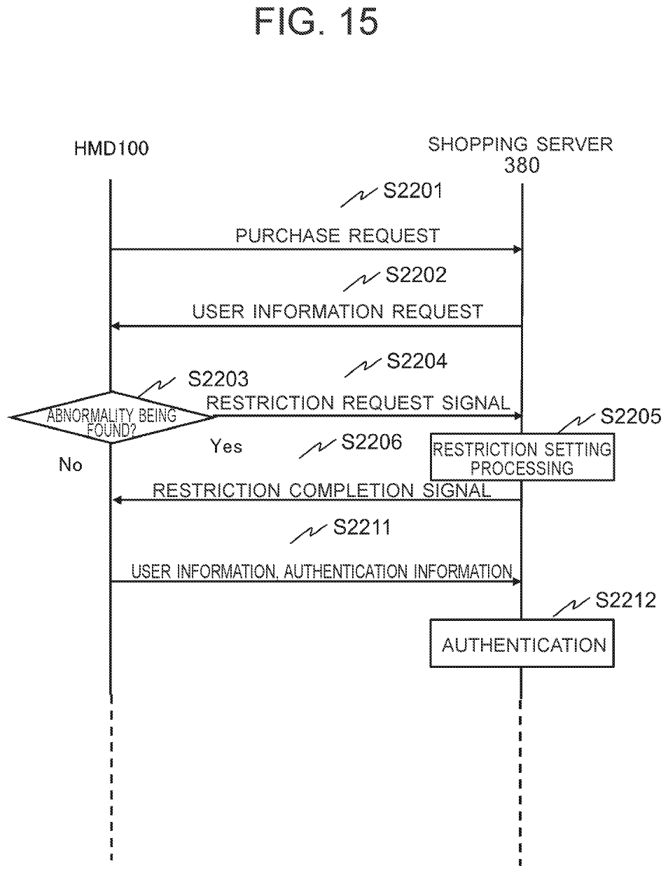

In the following, the present modification will be described with a specific example of transmission and reception of a signal between the HMD 100 and the shopping server 380. FIG. 15 is a diagram for explaining a flow of the processing according to the present modification. Hereinafter, the application for ΔΔ shopping installed in the HMD 100 is referred to as a shopping app.

The shopping app transmits a purchase request to the shopping server 380 (step S2201). In response thereto, the shopping server 380 transmits a user information request to the HMD 100 that is a transmission source of the purchase request (step S2202).

Upon detecting that the application to be monitored carries out the processing to be monitored has been performed, firstly, the controller 223 determines whether the abnormality information 234 is recorded (step S2203).

If the abnormality information 234 is recorded, the controller 223 transmits a restriction request signal to the shopping server 380 (step S2204). At this time, the shopping app records that the restriction request signal has been transmitted.

Upon receiving the restriction request signal, the shopping server 380 carries out the restriction setting processing (step S2205). The restriction setting processing is the processing of providing a predetermined restriction on access from the request source and informing the payment server 390 not to accept a payment request from the request source. The restriction includes, for example, not to accept a purchase confirmation request from the HMD 100 which is the request source.

Upon completion of the restriction setting processing, the shopping server 380 transmits a restriction completion signal to the shopping app (step S2206). The shopping app that has received the restriction completion signal continues the normal shopping processing. Here, for example, the shopping app transmits the user information and the authentication information to the shopping server 380 (step S2211), and receives the authentication (step S2212).

In this case, however, the shopping app cannot execute the processing restricted in step S2205. For example, the shopping app cannot confirm a purchase nor proceed to payment.

On the other hand, if the abnormality information 234 is not recorded, the shopping app continues the normal shopping processing of step S2211 and step S2212.

Thereafter, upon deletion of the abnormality information 234, the controller 223 transmits a restriction cancellation request to the server that has the record of transmission of the restriction request signal.

Note that the restriction cancellation request is not necessarily transmitted at this timing. For example, it may be transmitted, when the wearer 109 transmits the processing to be monitored (for example, purchase request) using the same shopping app on the HMD 100 thereafter. In this case, the controller 223 transmits a restriction cancellation request signal for requesting cancellation of the restriction when the abnormality information is not registered and the record of transmission of the restriction request signal is found.

Upon receiving the restriction cancellation request signal, the shopping server 380 carries out the restriction cancellation processing. The restriction cancellation processing is the processing of cancelling the restriction on access from the request source and also informing the payment server 390 of the cancellation.

The processing described above allows a shopping site (shopping server 380) to provide an appropriate restriction when the wearer 109 with the determination of “an abnormality is found” makes a purchase request to the shopping site. In addition, payments may also be restricted.

Before the controller 223 transmits the restriction request signal, the shopping app may transmit the user information about the wearer 109. This allows the shopping server 380 to determine the type of restriction and whether the payment is permitted based on the user information.

Furthermore, upon receiving the restriction request signal, the shopping server 380 may provide a test for confirmation to the wearer 109 of the HMD 100 which is the transmission source of the request. The test for confirmation is the same as that of the first embodiment.

The shopping server 380 transmits a question to the HMD 100 which is the request source. Then, the shopping server 380 waits for a reply from the HMD 100. The controller 223 displays the question as received on the display to accept an answer within a predetermined period of time. Upon receiving the answer within the predetermined period of time, the controller 223 transmits the answer as received to the shopping server 380. In case of not receiving the answer within the predetermined period of time, the controller 223 transmits the fact that the answer is not obtained to the shopping server 380.

The shopping server 380 determines whether the answer as received is correct, including no answer. When the answer is correct, the shopping server 380 does not carry out the restriction setting processing, otherwise, carries out the restriction setting processing.

Various modifications of the first embodiment, except for the modification in which whether an abnormality is found is determined by the batch processing, can be applied to the present embodiment. Rather, in the present embodiment, when it is determined “an abnormality is found”, it is not necessary to notify the wearer 109 of the abnormality being found.

### Third Embodiment

Next, a third embodiment according to the present embodiment will be described. In the present embodiment, when it is determined “an abnormality is found” in the electroencephalogram of the wearer 109, an application program (assistance application program; hereinafter, simply referred to as an assistance application) for assisting the action of the wearer 109 is automatically activated.

In other words, when it is determined “an abnormality is found”, the assistance application is started as there is a possibility that the wearer 109 has a difficulty in making decision about his or her own action. The assistance application to be started is set in advance depending on the actions of the wearer 109 at different times.

For example, in case that there is a suspicion of dementia, in other words, when it is determined “an abnormality is found” in the electroencephalogram and the wearer 109 is going out, a route navigation application is made started as the assistance application. Thus, the HMD 100 provides directions to guide the wearer 109, for example, even on the road or the like, where he or she daily passes without any navigation if being in the normal state. Providing an audio navigation together with a visual navigation causes the wearer 109 to easily notice that the navigation is provided.

When the wearer 109 is being awake at home and using an induction cooker in the kitchen, a home appliance control application is started as the assistance application in order to make the power of the induction cooker turned off. A navigation in the home may be provided.

Hereinafter, the present embodiment will be described focusing on the features different from those of the first embodiment.

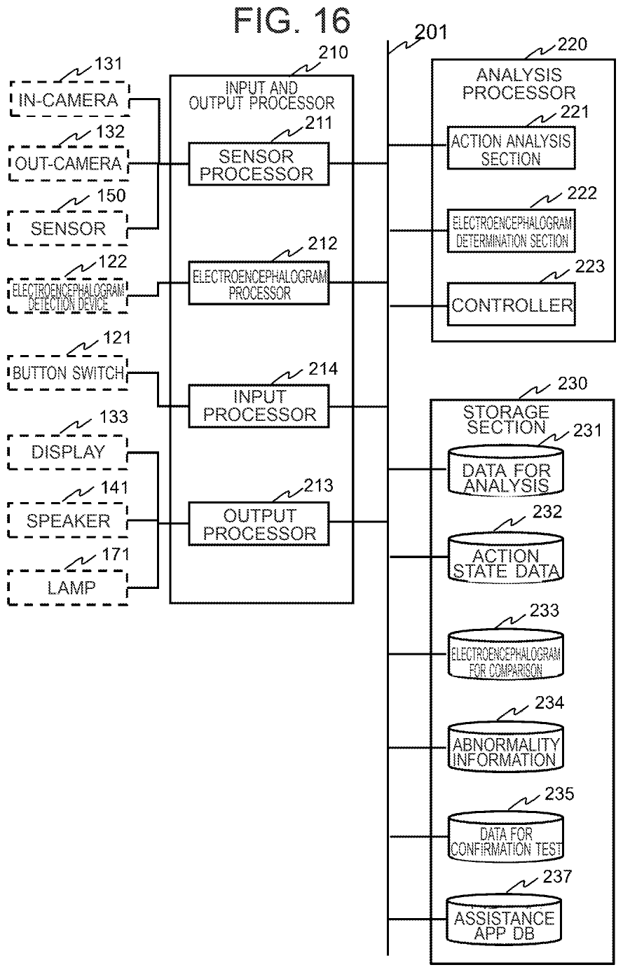

The appearance and hardware configuration of the HMD 100 according to the present embodiment are the same as those of the HMD 100 according to the first embodiment. The functional configuration according to the present embodiment is also the same as that according to the first embodiment. However, differences can be found in the processing of the action analysis section 221 and the controller 223, and also, as illustrated in FIG. 16, in an assistance application database (DB) 237, which will be described later, stored in the storage section 230 in advance for the processing by the controller 223.

The action analysis section 221 identifies, not only whether the wearer 109 is awake or sleeping, but also the current position of the wearer 109 when he or she is being awake. The current position to be identified shows, for example, whether the wearer 109 is out of home (going out) or at home, if being at home, in which room he or she is in, and the like. The result of identification is output to the controller 223. The action analysis section 221 identifies the current position, for example, based on a signal obtained from the GPS receiver 151. Furthermore, the action analysis section 221 identifies whether the wearer is at home or not based on the home address of the wearer 109.

When the electroencephalogram determination section 222 determines “an abnormality is found”, the controller 223 records the abnormality information 234 in the same manner as the first embodiment. Furthermore, the controller 223 carries out the processing of transmitting a startup signal (application startup processing) to a predetermined assistance application. Note that, in the application startup processing, when the electroencephalogram determination section 222 determines “no abnormality is found”, the controller 223 may carry out the processing of stopping the assistance application activated when it was determined “an abnormality is found”.

The assistance application to be activated in the application startup processing is set in advance for each current position of the wearer 109. For this setting, in the present embodiment, the assistance app DB 237 in which an assistance application to be started for each current position of the wearer 109 is registered is provided.

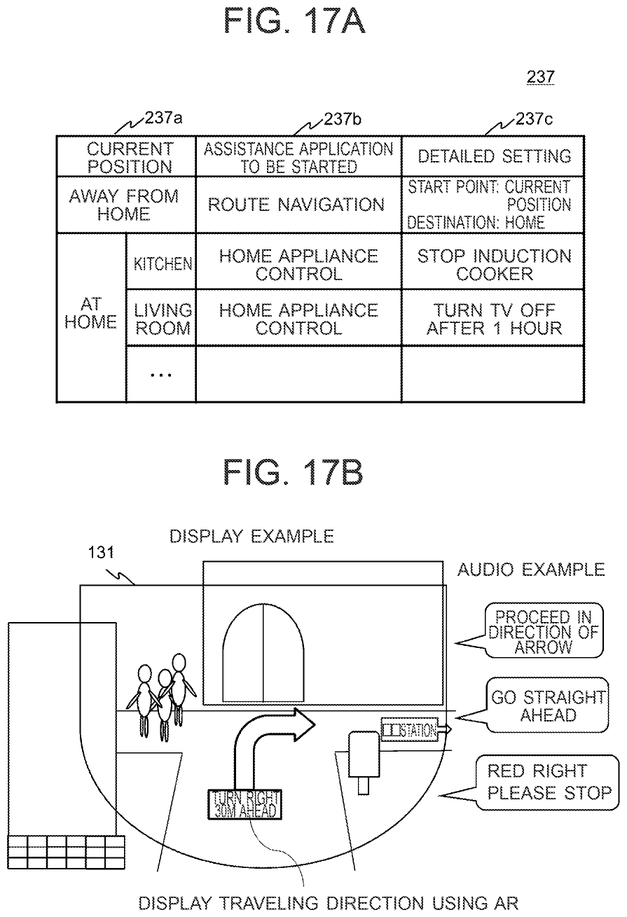

FIG. 17A illustrates an example of the assistance app DB 237. In the assistance app DB 237, an assistance application 237b to be started is registered for each current position 237a of the wearer 109. In addition, a detailed setting 237c to be set in the assistance application to be started is registered as necessary. The assistance application to be registered is the application installed in the HMD 100, which is for assisting the action of the wearer 109.

In this example, a route navigation application is registered as the assistance application 237b to be started for the case in which the wearer 109 is out, and as the detailed setting 237c, the home is registered as the destination. Note that different settings may be made depending on the distance between the current position and the home. For example, a different destination may be set. Specifically, in the case where the current position is away from the home by a predetermined distance or more, a public institution such as a nearby police box may be set as the destination.

The home appliance control application is registered as the assistance application 237b to be started for the case in which the wearer 109 is at home. For example, the detailed setting 237c, such as controlling the induction cooker to be stopped in the case where the current position is the kitchen and controlling the television to be turned off after one hour in the case where the current position is the living room, is registered.

For example, when it is determined “an abnormality is found” while the wearer 109 is out, the route navigation application is made started. FIG. 17B illustrates a display example on the display 133 and an audio output example to the speaker 141 in this case.

The route navigation application assists the wearer 109 by, for example, displaying the traveling direction using an AR technology. In addition, in the example illustrated in FIG. 17B, the route navigation application provides not only a visual route navigation on the display, but also provides an audio route navigation to assist the wearer 109.

### [Analysis and Evaluation Processing]

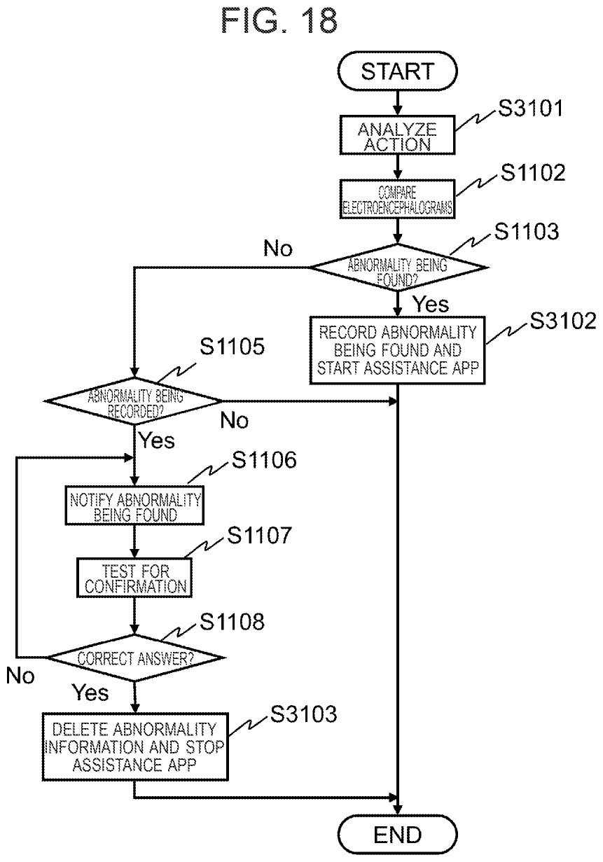

FIG. 18 illustrates a flow of the analysis and evaluation processing according to the present embodiment. This processing is carried out at predetermined time intervals in the same manner as the first embodiment. In the following, in the processing, differences from those of the first embodiment will be mainly described.

Firstly, in the present embodiment, in the action analysis, the action analysis section 221 acquires the current position information about the wearer 109 as well (step S3101).

When it is determined “an abnormality is found”, the controller 223 not only records the abnormality information 234, but also starts the assistance application set in the assistance app DB 237 (step S3102), and then ends the processing. In this processing, the controller 223 uses the information on the current position of the wearer 109 identified by the action analysis section 221. At this time, the controller 223 sets the parameters registered in the detailed setting 237c as necessary.

On the other hand, when it is determined “no abnormality is found”, the controller 223 determines whether the abnormality information 234 is recorded in the same manner as the first embodiment. If the abnormality information 234 is recorded, the controller 223 repeatedly provides the abnormality notification and the test for confirmation until the correct answer is obtained in the test for confirmation (step S1106, step S1107, step S1108). Upon obtaining the correct answer in the test for confirmation, the controller 223 deletes the abnormality information 234 and stops the assistance application being started (step S3103), and then ends the processing. If the assistance application is not being activated, the controller 223 deletes the abnormality information 234 and ends the processing.

As described above, in the HMD 100 according to the present embodiment, when the electroencephalogram determination section 222 determines “an abnormality is found”, the controller 223 starts a predetermined assistance application depending on the current position of the wearer 109.

As described above, the present embodiment enables appropriate assistance for the wearer 109 found to have an abnormality in his or her electroencephalogram.

### <Eleventh Modification>

In the embodiment described above, when the electroencephalogram determination section 222 determines “an abnormality is found”, the HMD 100 starts an assistance application. Here, as the assistance application, an application for recording action logs may be started. The action logs may be recorded, for example, only while the user is being awake.

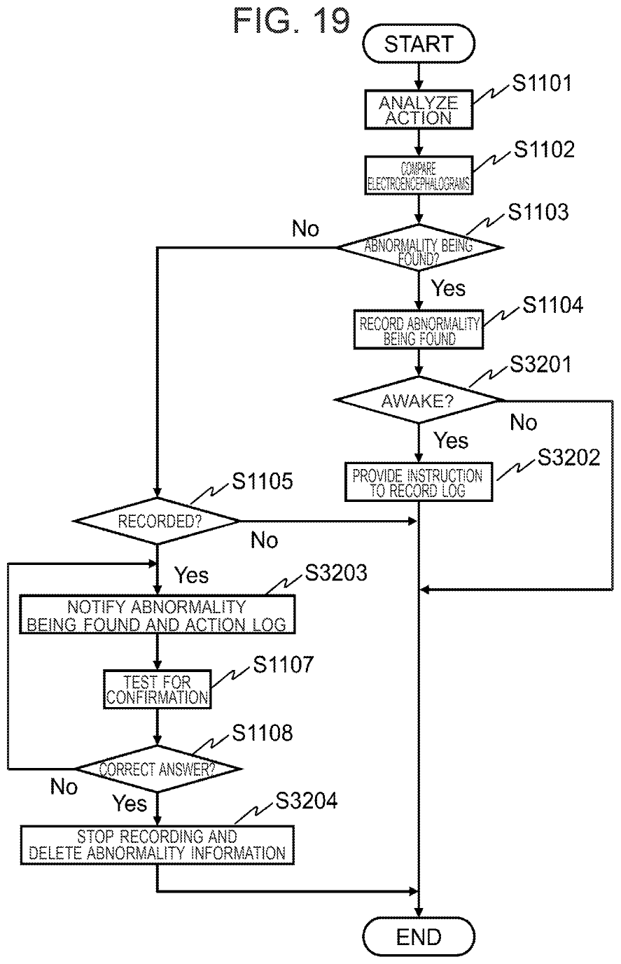

FIG. 19 illustrates a flow of the processing in this case. In the following, an example in which only whether the wearer 109 is awake is analyzed in the same manner as the first embodiment, and the action logs only while the wearer is being awake are recorded will be described.

As illustrated in FIG. 19, when the electroencephalogram determination section 222 determines “an abnormality is found”, the controller 223 records the abnormality information 234, and then determines whether the wearer 109 is awake (step S3201). In the case where the wearer 109 is awake, the controller 223 outputs an instruction to record action logs (step S3202). For example, the controller 223 outputs a startup instruction to an application for recording action logs. The controller 223 may output the startup instruction or an operation start instruction, not only to one application, but also a plurality predetermined applications capable of obtaining action logs, such as applications for recording audio data, video data, and operations, and applications for photographing. If the wearer 109 is not being awake, the controller 223 ends the processing without the action logs being recorded.

At the timing when it is determined “no abnormality is found”, in the abnormality notification processing, the controller 223 provides not only a notification that an abnormality has been found, but also the action logs (step S3203). Note that the controller 223 does not need to notify all the action logs at this time, and may notify only a predetermined action log. Furthermore, instead of notifying the action logs per se, the controller 223 may notify that the action logs are recorded.

FIG. 6C illustrates a display example 410b on the display 133 in this case. In the display example 410b, in addition to the display content of the display example 410, a message for notifying that the action logs are recorded, an icon 415, and the like are displayed. For example, the controller 223 may display the action logs upon receiving a selection of the icon 415 from the wearer 109.

Thereafter, the controller 223 provides a test for confirmation (step S1107), deletes the abnormality information 234 upon obtaining a correct answer, stops recording logs (step S3204), and ends the processing.

The action logs to be recorded are, for example, videos, audio data, or moving routes, which include the time. An application for recording action logs may be installed in advance so that all necessary information is recorded only by starting the application. The out-camera 132 may be started to record a video. The microphone 142 may be started to record audio data. For recording a moving route, the current position data received by the GPS receiver 151 may be recorded in association with the time.

In recording the audio data, for example, not only a conversation but also voices in a call may be recorded. The data obtained by recording may be stored as audio data or converted into text and stored as text data. In this case, an application for converting audio data into text which was installed in advance is made started together with the application for recording.

In recording the video, the out-camera 132 is used to acquire an image of the face of the other person. If an application for image recognition and verification is installed, the area corresponding to the face in the image of the face may be analyzed to identify the other person. This identification is carried out within the range of people registered in advance.

Analyzing the video as acquired may allow an object toughing the hand of the wearer 109, an object held in the hand of the wearer 109, an object that was held in the hand of the wearer 109 but taken off therefrom after a predetermined period of time, and the like to be identified.

In addition, as the operation information of the HMD 100, action logs of the operation records of the button switch 121 may be stored. In addition, in case that an impact of a predetermined value or more is given to the HMD 100, the time, the strength of the given impact, and the like may be recorded by processing the output of the acceleration sensor 154 or the like. Furthermore, in case that an impact of a predetermined value or more is given, instead of storing all the videos as recorded, the videos for a predetermined period of time before and after the impact may be stored.

As described above, according to the present modification, when the electroencephalogram determination section 222 determines “an abnormality is found”, the action logs of the wearer 109 in the abnormal state are collected and notified while he or she is in the normal state. This enables the wearer 109 to know his or her own action while his or her electroencephalogram shows an abnormality.

In the present modification, the action logs to be recorded may be made vary depending on the current position of the wearer 109. In this case, instead of the action analysis of step S1101, the action analysis of step S3101 is carried out to identify the current position of the wearer 109.

Furthermore, in the present modification, not only action logs are recorded, but also the assistance application registered in the assistance app DB 237 may be started.

Also in the present embodiment, various modifications of the first embodiment, except for the modification in which whether an abnormality is found is determined by the batch processing, can be applied to the present embodiment. Furthermore, the present modification may be combined with the second embodiment. In the present embodiment, when it is determined “an abnormality is found”, it is not necessary to notify the wearer 109 of an abnormality being found.

### Fourth Embodiment

In each of the embodiments and modifications described above, the example in which an abnormality in the electroencephalogram is caused by dementia has been described. However, the factor causing an abnormality in the electroencephalogram is not limited to dementia. For example, the electroencephalogram may be determined only when the factor causing an abnormality in the electroencephalogram occurs.

An example of an abnormality occurring in the memory function is drinking alcohol. When an abnormality occurs in the memory function due to drinking alcohol or the like, the electroencephalogram shows a waveform state similar to that of Alzheimer's dementia.

In the following, the case in which drinking alcohol causes an abnormality will be described as an example according to the present embodiment. In other words, in the HMD 100 according to the present embodiment, when it is determined that the wearer 109 has drunk alcohol, whether an abnormality is found in the electroencephalogram is determined at predetermined time intervals in the same manner as the embodiments described above. Then, when it is determined “an abnormality is found”, his or her actions thereafter are recorded. Then, the wearer 109 is notified of his or her actions when he or she is in the normal state. Note that the action may be recorded as an action log or in a video.

In the following, the present embodiment will be described focusing on the features different from those of the first embodiment.

The appearance and hardware configuration of the HMD 100 according to the present embodiment are the same as those of the HMD 100 according to the first embodiment. The functional configuration according to the present embodiment is also the same as that according to the first embodiment. However, differences can be found in the functions of each element.

The action analysis section 221 determines not only whether the wearer 109 is awake or sleeping, but also whether the wearer 109 has drunk alcohol. When the action analysis section 221 determines that he or she has drunk, a record of drinking is recorded.

For example, the action analysis section 221 analyzes the image acquired by the out-camera 132 to determine whether the wearer 109 has drunk alcohol. Specifically, the action analysis section 221 determines that the wearer 109 has drunk alcohol if drinking alcohol (bottle, can, container: glass, jug, cup, or the like) is detected based on the camera image in which the wearer 109 is captured.

Drinking alcohol may be determined based on the output from an alcohol detection sensor further included in the sensor 150. It may be also determined based on the schedule (welcome party, new year party, drinking party, or the like) of the wearer 109 registered in the HMD 100.

Drinking alcohol may be determined based on the report from the wearer 109. In this case, the controller 223 generates, for example, an input screen and displays it on the display 133 to accept the report from the wearer 109.

Upon receiving a notification that the wearer 109 has drunk alcohol from the action analysis section 221, the electroencephalogram determination section 222 according to the present embodiment starts determination of whether an abnormality is found in his or her electroencephalogram. In the same manner as the embodiments described above, the electroencephalogram determination section 222 makes the determination at predetermined time intervals.

When it is determined “an abnormality is found”, the controller 223 records the abnormality information 234 and provides an instruction to start recording action logs. The content to be recorded includes, for example, the same as those according to the modification of the third embodiment.

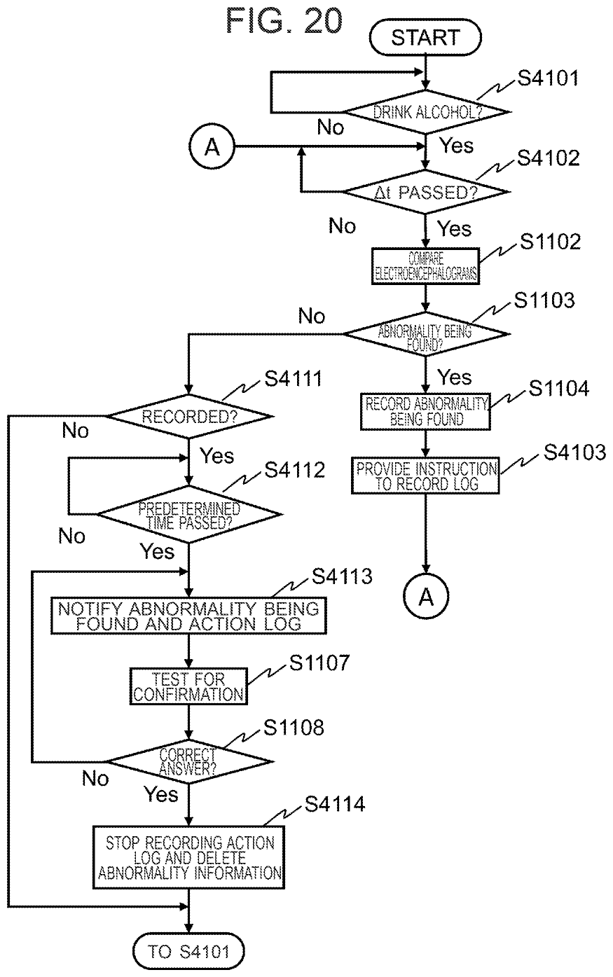

In the following, a flow of the processing by the HMD 100 according to the present embodiment will be described with reference to FIG. 20. This processing is started when the HMD 100 is activated.

Firstly, the action analysis section 221 detects whether the wearer 109 has drunk alcohol, at predetermined intervals (step S4101).

When detecting that the wearer 109 has drunk alcohol, the electroencephalogram determination section 222 matches the electroencephalograms at predetermined intervals (step S4102, step S1102).

When the electroencephalogram determination section 222 determines “an abnormality is found”, the controller 223 records the abnormality information 234 (step S1104), and outputs an instruction to record the action logs to the corresponding application or various functions (step S4103). The details are the same as those according to the modification of the third embodiment. Thereafter, the HMD 100 returns to step S4102 and repeats the processing from the determination processing.

On the other hand, if it is determined “no abnormality is found” in step S1103, the controller 223 determines whether the abnormality information 234 is recorded (step S4111).

If the abnormality information 234 is recorded, the controller 223 waits for elapse of a predetermined period of time from the timing at which it is determined “no abnormality is found” (step S4112). This allow the controller 223 to record the action logs for a certain period of time even after the state of the wearer 109 is changed from the state of “an abnormality is found” to the state of “no abnormality is found”.

After the predetermined period of time, the controller 223 notifies the wearer 109 of the abnormality information 234 and the action log as recorded. This is repeated until the correct answer is obtained in the test for confirmation (step S4113, step S1107, step S1108). Thereafter, the controller 223 stops recording of the action logs, deletes the abnormality information (step S4114), and returns to step S4101.

As described above, according to the HMD 100 of the present embodiment, the electroencephalogram determination section 222 starts determination of the electroencephalogram in response to a predetermined action such as drinking alcohol made by the wearer 109. When the electroencephalogram determination section 222 determines “an abnormality is found”, the wearer 109 is provided with the notification thereof while he or she is in the normal state. Furthermore, in the HMD 100 according to the present embodiment, when it is determined “an abnormality is found”, the action log of the wearer 109 is recorded, and the wearer 109 is notified of the log while his or her condition is normal.

The HMD 100 according to the present embodiment can appropriately detect that, for example, an abnormality such that a memory disfunction similar to dementia is suspected appears in the electroencephalogram of the wearer 109 due to a predetermined action such as drinking alcohol, and thus appropriately respond thereto. That is, according to the present embodiment, when the wearer 109 has drunk alcohol and an abnormality similar to a memory disfunction appears in his or her electroencephalogram, action logs are automatically recorded so as to be checked while the wearer 109 is in the normal state. This enables the wearer 109 to know his or her own actions while the memory disfunction occurs, and thus appropriately respond thereto as necessary.

In particular, in the present embodiment, action logs are recorded for a certain period of time even after the determination of “an abnormality is found” is changed to the determination of “no abnormality is found”. This allows action logs while the consciousness of the wearer 109 is ambiguous to be recorded as well even if his or her electroencephalogram shows no abnormality.

### <Twelfth Modification>

In the case of determining whether the wearer 109 has drunk alcohol based on the report from the wearer 109, the processing above is started after step S4101 in response to the report from the wearer 109.

Various modifications of the first embodiment, except for the modification in which whether an abnormality is found is determined by the batch processing, can be applied to the present embodiment as well.

### <Thirteenth Modification>

In each of the embodiments and modifications described above, an electroencephalogram is detected on the head. However, detection of the electroencephalogram is not limited thereto. In recent years, a technique of reading an electroencephalogram on the skin surface of the arm or hand, for example, Body Wave Technology, has been developed. For example, an electroencephalogram may be detected using a wearable terminal such as a smartwatch, a smartphone, or the like, which implements this technique.

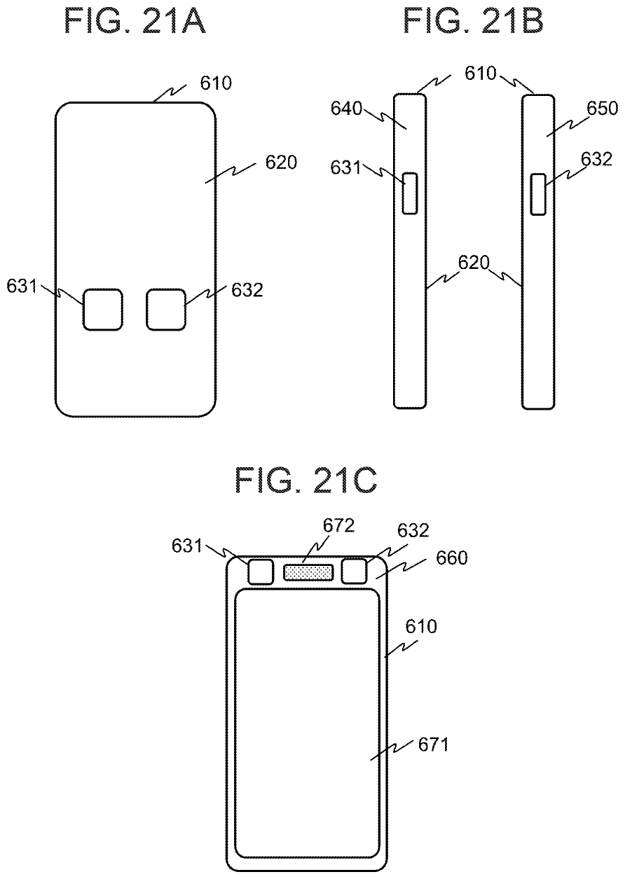

FIG. 21A and FIG. 21B illustrate an example of a smartphone 610 to which the technique as described above is applied.

FIG. 21A illustrates an example in which two electrodes for electroencephalogram measurement 631, 632 are placed on a back surface 620 of the smartphone 610. FIG. 21B illustrates an example in which the two electrodes for electroencephalogram measurement 631, 632 are placed on both side surfaces 640, 650 of the smartphone 610.

In this case, a bipolar lead method using a potential difference from two electrodes is employed for electroencephalogram measurement. The electrodes 631, 632 are placed on the portions of the back surface 620 or both the side surfaces 640, 650 which allow to be easily touched by the hand.

In the examples illustrated in FIG. 21A and FIG. 21B, the two electrodes 631, 632 are mounted, however, more than two electrodes may be arranged so that two portions touched by the hand are automatically selected and measured. Note that the back surface 620 is the surface opposite to the surface (front surface) on which the display is placed.

Note that the electrodes illustrated in FIG. 21A and FIG. 21B may be mounted, or they may be mixed and placed. The electrodes may be placed in a mixed manner, for example, on one position of the back surface 620 and one of the positions of the side surfaces 640, 650, respectively, or, on one position of the back surface 620 and two positions of the side surfaces 640, 650, respectively.

Furthermore, electrodes capable of detecting a potential in a non-contact state may be used. FIG. 21C illustrates an example of arrangement of the electrodes in this case. In this case, for example, the electrodes 631, 632 are placed at both the sides of a speaker 672 on a front surface 660 of the smartphone 610, where are the upper portions of a display 671.

For example, non-contact electrodes for Body Area Network have been developed. The example of arrangement above adopts the electrodes using this technology. In this case, bringing the speaker 672 on the front surface 660 of the smartphone 610 close to the ear when there is an incoming call enables an electric potential of the electroencephalogram of the user to be introduced. In this case, these non-contact electrodes do not have to be in contact with the head.

Each of the electrodes 631, 632 includes an amplifier, an ADC, and a communication module, and can transmit the potential from one electrode to the other electrode. In the case of the electrodes 631, 632, the potential can be introduced even if there is an insulator between each of the electrode 631, 632 and the head.

### <Fourteenth Modification>

Furthermore, in the case where an abnormality to be detected is dementia, the abnormality may be determined based on information other than an electroencephalogram. For example, an abnormality may be determined using a facial expression of the wearer 109 (user), in particular, using the expressions and movement of the eyes. For example, the condition of the brain is normal or abnormal is determined based on the state of the eyes of the wearer 109 tracking a moving target object displayed on the display section (display). The target object is, for example, a pointer, an icon, or the like. The tracking condition of the eyes of the wearer 109 is determined by analyzing images of the eyes of the wearer 109 acquired by the in-camera 131. For this analysis, any method can be employed.

In this case, the target object is displayed, for example, periodically during awake. Alternatively, the target object may be started displaying after a resting state, such as while the wearer 109 is sitting down, continues for a certain period of time. Making the target object blink or providing an audio notification enables the wearer 109 to easily know that the target object is being displayed.

### <Fifteenth Modification>

In each of the embodiments and modifications described above, it sometimes may be difficult to accurately measure an electroencephalogram depending on the action state during awake. Electroencephalogram measurement may not be carried out while a person who is a target of measurement is moving at a certain degree or more. In such a case, as the electroencephalograms 233b, 233c of the electroencephalogram for comparison 233, the ones obtained while the target of measurement is moving within a predetermined range are used.

For example, the maximum value of the amount of motion for acquiring electroencephalogram data may be recorded in the electroencephalogram for comparison 233 in a predetermined unit of measurement. The action analysis section 221 may refer to the maximum value and determine that the determination cannot be made when the motion of the wearer 109 as detected is equal to or more than the maximum value.

In the embodiments and modifications described above, the HMD 100, the smartphone 610, the smartwatch, and the like have been exemplified, however, the present invention is not limited thereto. The present invention can be applied to a mobile information terminal having at least the functions of acquiring an electroencephalogram of a user and notifying it.

Furthermore, the present invention is not limited to the embodiments described above, and includes various modifications. For example, the embodiments described above have been described in detail for the purpose of clarifying the present invention, and the present invention is not limited to those having all the features as described. In addition, a part of the configuration of the present embodiments can be replaced with that of other embodiments, and the features of other embodiments and modifications can be added to the configuration of the present embodiments. Furthermore, it is possible to add, delete, or replace other configurations with respect to a part of the configuration of the present embodiments.

Some or all the configurations, functions, processing units, and processing means described above may be implemented by hardware, for example, by designing them with an integrated circuit. In addition, the configurations and functions described above may be implemented by software by interpreting and executing programs in which the processor implements the respective functions. Information such as programs, tables, and files for implementing various functions can be placed in recording devices such as a memory, hard disk, and solid-state drive (SSD), or recording media such as an IC card, SD card, and DVD.

Furthermore, the control lines and information lines which are considered to be necessary for the purpose of explanation are indicated herein, but not all the control lines and information lines of actual products are necessarily indicated. It may be considered that almost all the configurations are actually connected to each other.

### REFERENCE SIGNS LIST

100: HMD, 101: main processor, 102: system bus, 109: wearer, 110: memory and storage, 111: RAM, 112: ROM, 113: flash memory, 120: input I/F, 121: button switch, 122: electroencephalogram detection device, 130: image processing device, 131: in-camera, 132: out-camera, 133: display, 140: audio processing device, 141: speaker, 142: microphone, 150: sensor, 151: GPS receiver, 152: geomagnetic sensor, 153: range sensor, 154: acceleration sensor, 155: gyro sensor, 156: biometric information acquisition sensor, 160: communication I/F, 161: wireless communication I/F, 162: telephone network communication I/F, 171: lamp, 172: extended I/F, 173: timer, 180: main body portion, 190: support portion,
201: bus, 210: input and output processor, 211: sensor processor, 212: electroencephalogram processor, 213: output processor, 214: input processor, 220: analysis processor, 221: action analysis section, 222: electroencephalogram determination section, 223: controller, 230: storage section, 231: data for analysis, 232: action state data, 232a: time, 232b: state, 233: electroencephalogram for comparison, 233a: state, 233b: electroencephalogram in normal state, 233c: electroencephalogram in abnormal state, 234: abnormality information, 234a: time, 234b: determination result, 234c: notification column, 235: data for confirmation test, 236: instruction setting, 236a: name, 236b: restriction for abnormality, 236c: app support, 236d: processing to be monitored, 237: assistance app DB, 237a: current position, 237b: assistance application, 237c: detailed setting,
310: network, 320: server, 330: wireless router, 340: storage device, 360: smartphone, 370: smartwatch, 380: shopping server, 390: payment server,
412: message, 413: confirmation button display, 414: message, 415: icon, 421: icon, 422: icon, 423: icon, 610: smartphone, 620: back surface, 631: electrode for electroencephalogram measurement, 632: electrode for electroencephalogram measurement, 640: side surface, 650: side surface, 660: front surface, 671: display, 672: speaker

1. A user assistance system comprising:
an electroencephalogram measurement device configured to output electroencephalogram data on a user;
an action measurement device configured to output action data on the user;
an electroencephalogram determination device configured to determine whether an abnormality is found in the electroencephalogram data based on the electroencephalogram data and the action data; and
a controller configured to carry out predetermined processing in response to a result of determination indicating that the abnormality has been found.

2. The user assistance system according to claim 1, further comprising:
an action analysis device configured to determine whether the user is awake based on the action data; and
a storage device, wherein
the controller is configured to:
in response to the result of determination that the abnormality has been found, store abnormality information indicating the abnormality has been found in the storage device; and
in a case where the abnormality information is stored in the storage device, provide an abnormality notification for notifying that the abnormality has been found in the electroencephalogram data and delete the abnormality information in a period in which the result of determination indicates that the user is awake and also the result of determination indicates that no abnormality is found.

3. The user assistance system according to claim 2, wherein
the controller provides a predetermined test for confirmation after providing the abnormality notification and before deleting the abnormality information, and repeatedly provides the abnormality notification and the test for confirmation until the controller receives a correct answer from the user.

4. The user assistance system according to claim 1, wherein
in response to the result of determination indicating the abnormality has been found, the controller transmits a restriction signal for restricting processing to a predetermined application program.

5. The user assistance system according to claim 4, further comprising a communication interface, wherein
the controller transmits the restriction signal to a predetermined external device via the communication interface.

6. The user assistance system according to claim 1, further comprising a communication interface, wherein
in response to the result of determination indicating that the abnormality has been found, the controller transmits a restriction request signal for requesting a restriction of processing to a server that is a service provider of a predetermined application program via the communication interface.

7. The user assistance system according to claim 1, wherein
in response to the result of determination indicating that the abnormality has been found, the controller transmits a startup signal to a predetermined assistance application program.

8. The user assistance system according to claim 1, wherein
the controller collects action logs of the user in response to the result of determination indicating that the abnormality is found.

9. The user assistance system according to claim 1, wherein
the electroencephalogram determination device starts the determination based on the action data in response to a predetermined action performed by the user.

10. A wearable terminal comprising:
an electroencephalogram measurement section configured to output electroencephalogram data on a user;
an action measurement section configured to output action data on the user;
an electroencephalogram determination section configured to determine whether an abnormality is found in the electroencephalogram data based on the electroencephalogram data and the action data; and
a controller configured to carry out predetermined processing in response to a result of determination indicating the abnormality has been found.

11. A user assistance method for a wearable terminal, the wearable terminal comprising:
an electroencephalogram measurement section configured to output electroencephalogram data on a user;
an action measurement section configured to output action data on the user; and
an information processor configured to carry out processing using the electroencephalogram data and the action data,
the method comprising:
determining whether an abnormality is found in the electroencephalogram data based on the electroencephalogram data and the action data; and
carrying out predetermined processing in response to a result of determination indicating the abnormality has been found.

12. (canceled)
